(19) United States
(12) Patent Application Publication
(10) Pub. No.: US 2003/0157527 A1
(43) Pub. Date: Aug. 21, 2003

(54) Identification of rearrangements in nucleic acid molecules

(75) Inventor: Rusla Marianne Dubreuil Lastrucci, Huntsville, AL (US)

Correspondence Address: STERNE, KESSLER, GOLDSTEIN & FOX PLLC, 1100 NEW YORK AVENUE, N.W., WASHINGTON, DC, 20005, US

(73) Assignee: Invitrogen Corporation

(21) Appl. No.: 10/308,162

(22) Filed: Dec. 3, 2002

Related U.S. Application Data
(60) Provisional application No. 60/357,631, filed on Feb. 20, 2002.
(60) Provisional application No. 60/341,863, filed on Dec. 21, 2001.
(60) Provisional application No. 60/334,531, filed on Dec. 3, 2001.

(51) Int. Cl. C12Q 1/68

(52) U.S. Cl. 435/6

(57) ABSTRACT

Disclosed are methods and compositions for detecting a chromosomal rearrangement in a sample of nucleic acids. In an exemplary method, a first nucleic acid comprising a portion of a first chromosome, which may be detectably labeled, is attached to a substrate; a first portion of a test nucleic acid is hybridized to the first nucleic acid; a second nucleic acid comprising all or a portion of a second chromosome, which may be detectably labeled, is hybridized to a second portion of the test nucleic acid, thereby forming a trimolecular sandwich, and the hybridization of the test nucleic acid to both of the first and second nucleic acids is detected as an indication that the test nucleic acid comprises a chromosomal rearrangement. In particular embodiments, the first and second nucleic acids are derived from the same chromosome. In a related method, the test nucleic acid is used as a template for nucleic acid synthesis, and primers derived from a first and a second chromosome or from the same chromosome, which are distinctly labeled with first and second labels, respectively, are used to prime nucleic acid synthesis. A synthesized nucleic acid comprising each of the first and second primers is detected as an indication that the test nucleic acid comprises a chromosomal rearrangement. Also disclosed are kits for detecting chromosomal rearrangements. Such methods and kits can be used, for example, in the diagnosis or identification of disease-associated chromosomal rearrangements, e.g., cancers such as leukemia.

### CROSS-REFERENCE TO RELATED APPLICATIONS

This application claims priority under 35 U.S.C. §119(e) from U.S. provisional application Nos. 60/334,531; 60/341,863; and 60/357,631, filed Dec. 3, 2001; Dec. 21, 2001; and Feb. 20, 2002; respectively, each of which is incorporated herein by reference.

### BACKGROUND OF THE INVENTION

1. Field of the Invention

The present invention relates to methods and compositions for identifying chromosomal rearrangements.

2. Related Art

The rearrangement of nucleic acid molecules (e.g., chromosomal rearrangements such as translocations, deletions, inversions, or fusions) can result in disorders such as cancers, including solid tumors and non-solid cancers such as leukemias and lymphomas. For example, the Philadelphia chromosome (Ph) in chronic myeloid leukemia (CML) involves a reciprocal translocation, t(9;22)(q34;q11), bringing the 3′ c-abl proto-oncogene sequences of chromosome 9 adjacent to the 5′ sequences of the 5.8 kb breakpoint cluster region (BCR) on chromosome 22. This hybrid gene is transcribed into two forms of 8.5 kb chimeric BCR-abl mRNA, which differ by 75 bp. These mRNAs are translated into a chimeric 210 kd-protein product that is considered essential to the pathogenesis of CML. Other cancers, including other leukemias and lymphomas, can also result from chromosomal translocations. Several examples of cancer-associated chromosomal translocations are described herein. Conventional methods for identifying chromosomal translocations include classical cytogenetic analysis, fluorescence in situ hybridization (FISH), and PCR.

### SUMMARY OF THE INVENTION

The invention provides methods and compositions for detecting rearrangements (e.g., translocations, deletions, inversions, or fusions) in nucleic acid molecules such as chromosomes. Thus, in one aspect, the invention provides a method for identifying one (e.g., at least 1, 2, 3, 4, 5, 6, etc.) rearrangements (e.g., chromosomal rearrangements) in a sample which contains a nucleic acid, i.e., a “test” nucleic acid (e.g., a sample obtained from a patient), as well as compositions for performing such a methods. The invention further provides methods and compositions for identifying differences (e.g., structural differences) between nucleic acids (e.g., chromosomal nucleic acids) contained in two or more (e.g., 2, 3, 4, 5, 6, 7, 8, etc.) samples, as well as reaction mixtures prepared upon combining materials for performing methods of the invention.

The invention further provides methods and compositions for detecting structural differences between nucleic acid molecules, as well as reaction mixtures useful during the practice of methods of the invention. In particular embodiments, the invention provides methods for comparing the structures of two nucleic acid molecules. For example, one plasmid comprising nucleic acid regions A, B and C may recombine with another plasmid comprising nucleic acid regions A, D and E to yield a recombined plasmid comprising regions A, B, C, and E. In such an instance, methods of the invention may be used to detect the recombined plasmid, as well as the unrecombined starting plasmids.

Methods of the invention include those which comprise providing a substrate having attached thereto at least one first nucleic acid (a “capture” probe) comprising the sequence of all or a portion of a first chromosome or other nucleic acid molecule (e.g., a plasmid); contacting the substrate with a test nucleic acid under conditions that permit a first portion of the test nucleic acid to hybridize to all or a portion of the first nucleic acid; contacting the test nucleic with a second nucleic acid (a “detection” probe) under conditions that permit hybridization of all or a portion of the second nucleic acid to a second portion of the test nucleic acid, wherein the second nucleic acid comprises the sequence of all or a portion of a second chromosome or other nucleic acid molecules (e.g., a plasmid); and detecting hybridization of the test nucleic acid to each of the first nucleic acid and the second nucleic acid as an indication that the test nucleic acid comprises a rearrangement, e.g., a chromosomal translocation or other structural variation.

In one embodiment, the first nucleic acid is detectably labeled with a first label. In another embodiment, the second nucleic acid is detectably labeled with a second label. Generally, the second label is distinct from the first label. The colocalization of the first and the second labels can be detected as an indication that the test nucleic acid hybridizes to each of the first nucleic acid and the second nucleic acid. The invention further provides compositions and kits for performing the methods described herein, as well as a reaction mixture prepared upon combining materials for performing methods of the invention.

Optionally, hybridization of the test nucleic acid to the first and second nucleic acids can be detected by isolating the second nucleic acid following hybridization of the second nucleic acid to the test nucleic acid. For example, the test nucleic acid is contacted with the second nucleic acid. Non-hybridized second nucleic acid then is removed (e.g., washed away). Finally, the hybridized second nucleic acid is isolated (e.g., by increasing the temperature of the sample to denature the hybridizing nucleic acid), and the second nucleic acid is cloned into a vector and/or sequenced. Conventional molecular biology techniques can be used in such methods. Such methods do not necessitate that either the first or second nucleic acid be detectably labeled, although the nucleic acids may be labeled.

The substrate may be any solid support, such as a microchip, a microtiter plate, plastic, a nylon membrane, glass, or chromatography resin. The test nucleic acid and/or the nucleic acid comprising the sequence of all or a portion of a first or second chromosome or other nucleic acid molecule may comprise DNA or RNA. If desired, such nucleic acids may include synthetic nucleotides, e.g., to increase nucleic acid stability or hybridization.

The test nucleic acid may be derived from any natural or artificial source, and may include a naturally-occurring or artificial sequence (e.g., a synthetically produced nucleic acid molecule, a nucleic acid molecule which is assembled in vitro from segments, etc). Typically, the test nucleic acid is derived from a cell of an organism such as a cell of a multicellular eukaryote such as a human, mouse, chimpanzee, rat, dog, or plant (e.g., rice, wheat, corn, etc.), or a cell of a single cell organism, such as a bacterium, protozoan or fungus. The cell may be a primary cell (e.g., a primary human cell), or it may be a cell of a cell line (e.g., a human cell line).

The first and the second nucleic acids may be detectably labeled with a first and a second label, respectively, and the second label may be distinct from the first label. Examples of labels that can be used to label the first and/or second nucleic acids include, without limitation, enzymatic labels, fluorescent labels, radioisotopic labels (e.g., P³²), chemiluminescent labels, or antigenic labels.

Optionally, the first and/or second nucleic acid can be included within a vector. For example, the first and/or second nucleic acid may be a band-specific probe (such as those described by Invitrogen Corp., Carlsbad, Calif.) or a similar portion of chromosomal nucleic acid contained within a vector, a bacterial artificial chromosome (BAC), a yeast artificial chromosome (YAC), or the like. In carrying out the detection methods described herein, vector sequences can be blocked, if desired, by specifically or non-specifically hybridizing nucleic acids to the vector sequences (e.g., by using conventional methods to hybridize salmon sperm DNA, herring sperm DNA, or the like thereto). Alternatively, the vector sequences can be removed using conventional molecular biology techniques. Similarly, repetitive sequences in the chromosomal DNA can be blocked using conventional methods, such as with the use of Cot DNA, for example.

Optionally, the substrate can have attached thereto a population of distinct first nucleic acids (optionally with like molecules localized in discreet subsections of the substrate), wherein each first nucleic acid comprises the sequence of all or a portion of a chromosome or other nucleic acid molecule, and wherein the population of first nucleic acids comprises the sequences of all or a portion of a plurality (e.g., 2, 3, 4, 5, 6, 7, 8, 9, 10, 11, 12, 13, 14, 15, 16, 17, 18, 19, 20, 21, 22, 23, 24, or more) of chromosomes or other nucleic acid molecules. Similarly, the test nucleic acid can be contacted with a population of distinct second nucleic acids, wherein each second nucleic acid comprises the sequence of all or a portion of a chromosome or other nucleic acid molecule, and wherein the population of second nucleic acids comprises the sequences of all or a portion of a plurality (e.g., 2, 3, 4, 5, 6, 7, 8, 9, 10, 11, 12, 13, 14, 15, 16, 17, 18, 19, 20, 21, 22, 23, 24, or more) of chromosomes or other nucleic acid molecules. If desired, the population of first and/or second nucleic acids may be contained within a library. For example, a library of nucleic acids may be used as capture probes, with the library including a plurality of portions of a chromosome. If desired, such a library may include nucleic acids that correspond to a particular portion of a chromosome, e.g., a library of chromosome band-specific probes. Any of a variety of vectors can be used to prepare such a library, e.g., BACs, YACs, and the like.

The first and second nucleic acids may be distributed on the substrate so as to form a two-dimensional matrix, if desired. Such a matrix may include a plurality of subsections of the substrate, with each subsection having attached thereto a first nucleic acid. Thus, a plurality of hybridization reactions as described herein may be carried out simultaneously or non-simultaneously on a plurality of subsections of a substrate. For example, a population of distinct first nucleic acids (comprising all or a portion of a chromosome or other nucleic acid molecule) may be attached to a substrate, with sets of each distinct first nucleic acid forming a column(s) of spots of nucleic acid on the substrate (e.g., 0.1 to 3,000,000 pg of nucleic acid per subsection, with each spot in a diameter of 0.01 mm² to 100 mm²). The test nucleic acid then is hybridized to the first nucleic acid. Finally, a population of distinct second nucleic acids is allowed to contact the test nucleic acids, with each distinct second nucleic acid being applied to a distinct row of the substrate, thereby forming a two-dimensional matrix (see, e.g., FIG. 7). The matrix thus includes a plurality of subsections, and generally from about 2 to about60,000 rows and/or columns (e.g., at least 2, 4, 10, 20, 21, 22, 23, 23, 24, 50, 100, 200, 300, 400, 500, 1,000, 5,000, 10,000, 20,000, 30,000, or even 60,000 rows and/or columns; 2 to 5, 2 to 10; 2 to 20, 2 to 50, 2 to 75, 2 to 100, 2 to 1,000, 2 to 5,000, 2 to 10,000, 2 to 20,000, 2 to 30,000, 2 to 50,000, 5 to 10, 5 to 20, 5 to 50, 5 to 75, 5 to 100, 5 to 1,000, 5 to 5,000, 5 to 10,000, 5 to 20,000, 5 to 30,000, or 5 to 50,000 rows and/or columns). In many embodiments, each subsection of the matrix utilizes a distinct combination of capture and detection probes. In an alternative embodiment, different groups of first nucleic acid molecules are attached to a plurality of substrates. For example, various band-specific probes, corresponding to various chromosomes, are attached to a plurality of substrates. For example, first nucleic acids corresponding to a first chromosome may be attached to a first substrate; first nucleic acids corresponding to a second chromosome may be attached to a second substrate, etc.

The detection of a chromosomal rearrangement can be indicative, and thus diagnostic, of a disorder. Many inherited disorders result from chromosomal deletions, which can be identified using the methods described herein. Chromosomal translocations can result in cancers, such as solid tumors (e.g., carcinomas, adenomas, sarcomas), or non-solid cancers, such as leukemia or lymphomas. For example, nucleic acids comprising the following breakpoints of chromosomal translocations are indicative of various cancers: t(14;18) (follicular lymphoma); t(8;21) (acute myelogenous leukemia); t(9;22) (chronic myeloid leukemia/Ph′); t(15;17) (acute promyelocytic anemia); t(8;14) (Burkitt's lymphoma); t(11;14) (mantle cell lymphoma); t(9;14) (lymphoplasmacytoid lymphoma); t(5;14) (B-cell malignancy); t(1;14) (B-cell malignancy); t(3;14) (B-cell malignancy); t(14;15) (B-cell malignancy); t(1;14) (B-cell malignancy); t(4;14) (B-cell malignancy); t(6;14) (B-cell malignancy); t(14;16) (B-cell malignancy); t(2;5) (lymphoma); t(14;15) breast adenocarcinoma; t(3;8) salivary gland adenoma; t(9;16) skin basal cell carcinoma; t(3;5) bladder transitional cell carcinoma; t(2;13) alveolar rhabdomyosarcoma; and t(11;22) askins tumor, ewings sarcoma, and esthesioneuroblastoma. Accordingly, the invention provides methods for diagnosing a chromosomal abnormality in a patient (e.g., diagnosing cancer or a predisposition to cancer) by identifying a chromosomal translocation in a test nucleic acid derived from a patient, as described herein. Similarly, the invention also provides methods for determining the prognosis, progression, or treatment protocol of a cancer in a patient, which methods include identifying a chromosomal rearrangement in a test nucleic acid derived from a patient, as described herein.

In a related aspect, the invention provides a trimolecular sandwich comprising a substrate; a first nucleic acid comprising the sequence of all or a portion of a first chromosome or other nucleic acid molecule, wherein the first nucleic acid is attached to the substrate; a test nucleic acid, wherein a first portion of the test nucleic acid is hybridized to all or a portion of the first nucleic acid; a second nucleic acid, wherein all or a portion of the second nucleic acid is hybridized to a second portion of the test nucleic acid, and wherein the second nucleic acid comprises the sequence of all or a portion of a second chromosome or other nucleic acid molecule; wherein the first nucleic acid is optionally detectably labeled with a first label; and further wherein the second nucleic acid is optionally detectably labeled with a second label and wherein the second label is distinct from the first label. In particular embodiments, the substrate may not be present. The invention further includes methods for preparing the above-described trimolecular sandwich as well as reaction mixtures which contain such a sandwich and the use of such a sandwich to detect nucleic acid rearrangements.

Optionally, the substrate, when present, has attached thereto a population of distinct first nucleic acids, wherein each first nucleic acid comprises the sequence of all or a portion of a chromosome or other nucleic acid molecule, and wherein the population of first nucleic acids comprises the sequences of all or a portion of a plurality of chromosomes or other nucleic acid molecule. A mixed population of trimolecular sandwiches can comprise a population of distinct test nucleic acids. Similarly, a mixed population of trimolecular sandwiches can comprise a population of second nucleic acids, wherein each second nucleic acid comprises the sequence of all or a portion of a chromosome or other nucleic acid molecules, and wherein the population of second nucleic acids comprises the sequences of all or a portion of a plurality of chromosomes or other nucleic acid molecules. The test nucleic acid may include, for example, the breakpoint of a chromosomal translocation selected from the group consisting of t(14;18), t(8;21), t(9;22), t(15;17), t(8;14), t(11;14), t(9;14), t(5;14), t(1;14), t(3;14), t(14;15), t(1;14), t(4;14), t(6;14), t(14;16), t(2;5), t(14;15), t(3;8), t(9;16), t(3;5), t(2;13), and t(11;22).

In a related aspect, the invention also provides a method for identifying a test nucleic acid comprising a chromosomal rearrangement (e.g., a translocation) or other structural variation relative to the arrangement or structure of nucleic acids in a reference nucleic acid (e.g., a chromosome). The method includes providing a test nucleic acid as a template for nucleic acid synthesis; contacting the test nucleic acid with each of (i) a population of first primers, wherein substantially all of the first primers are detectably labeled with a first label, and wherein the population of first primers is randomly generated, and each of the first primers hybridizes to a portion of a first chromosome or other nucleic acid molecule and (ii) a population of second primers, wherein substantially all of the second primers are detectably labeled with a second label, and wherein the second primers are randomly generated, and each of the second primers hybridizes to a portion of a second chromosome or other nucleic acid molecule, and further wherein the second label is distinct from the first label. Methods of the invention further include those which involve (i) synthesizing a nucleic acid complementary to the test nucleic acid, wherein the test nucleic acid serves as a template for nucleic acid synthesis and one of the first and one of the second primers prime synthesis of the synthesized nucleic acid; and (ii) detecting a synthesized nucleic acid comprising each of the first and the second labels as an indication that the test nucleic acid comprises a chromosomal translocation or other nucleic acid structural variation. Optionally, the method can also include isolating the synthesized nucleic acid comprising each of the first and the second labels, e.g., by affinity chromatography. If desired, such affinity chromatography may utilize the label used to detectably label the first and/or second primer (e.g., a nucleic acid labeled with biotin can be bound to a solid support having streptavidin attached thereto). The invention further provides compositions and kits for performing the above methods.

The invention provides kits, which can be used to detect chromosomal rearrangements or differences between chromosomes as well as structural variations between other types of nucleic acid molecules present in samples (e.g., different organisms of different strains or species). Kits of the invention may include any one or more of the following components: (i) one or more (e.g., 1, 2, 3, etc) substrates (e.g., 1, 2, 3, 4, 5, or more) optionally having attached thereto a first nucleic acid comprising all or a portion of a first chromosome or other nucleic acid molecule, wherein the first nucleic acid is optionally detectably labeled with a first label; (ii) instructions for using the kit (e.g., instructions for using the kit to detect chromosomal rearrangements or differences between nucleic acids in accordance with the methods disclosed herein), (iii) one or more nucleic acid primers, e.g., degenerate oligonucleotide primers; and (iv) one or more second nucleic acids comprising all or a portion of a second chromosome or other nucleic acid molecule, wherein the second nucleic acid is optionally detectably labeled with a second label and wherein the second label is distinct from the first label. In certain embodiments, the substrate of kits of the invention has attached thereto a population of distinct first nucleic acids, wherein each of the first nucleic acids comprises the sequence of all or a portion of a chromosome or other nucleic acid molecule, and wherein the population of first nucleic acids comprises the sequences of all or a portion of a plurality of chromosomes or other nucleic acid molecules. In addition, or alternatively, the kit includes a population of second nucleic acids, wherein each of the second nucleic acids comprises the sequence of all or a portion of a chromosome, and wherein the population of second nucleic acids comprises the sequences of all or a portion of a plurality of chromosomes or other nucleic acid molecules. The kit may be compartmentalized and include containers that contain reagents of the kit (e.g., the substrate with the first nucleic acid).

The invention further includes kits for preparing substrates suitable for use with the present invention. Thus, kits of the invention may comprise one or more of the following components: (i) one or more (e.g., about 1, about 2, about 3, about 4, about 5, about 6, about 7, about 8, about 9, about 10, about 15, about 20, about 25, about 30, about 35, about 40, about 50, about 60, about 70, about 80, about 90, about 100, etc.) first nucleic acid molecule(s); (ii) one or more substrates to which modified and/or unmodified nucleic acid molecules may be attached; and (iii) instructions for preparing substrates which contain first nucleic acid molecules located at discreet locations. Additional components which may be included in kits of the invention include components described above and elsewhere herein, such as a second nucleic acid molecule(s).

The methods of the invention also can be used to identify chromosomal rearrangements or other structural variations involving a single chromosome or other nucleic acid molecule, such as chromosomal inversions, chromosomal fusions, and chromosomal deletions. In particular embodiments, the methods involve providing a substrate having attached thereto a first nucleic acid comprising the sequence of a first portion of a chromosome or other nucleic acid molecule; contacting the substrate with a test nucleic acid under conditions that permit a first portion of the test nucleic acid to hybridize to all or a portion of said first nucleic acid; contacting the test nucleic with a second nucleic acid under conditions that permit hybridization of all or a portion of the second nucleic acid to a second portion of the test nucleic acid, wherein the second nucleic acid comprises the sequence of a second portion of said chromosome or other nucleic acid molecule; and detecting hybridization of the test nucleic acid to each of the first nucleic acid and the second nucleic acid as an indication that the test nucleic acid comprises a chromosomal rearrangement or other structural variation. As described above, the first and/or second nucleic acid may be detectably labeled. In particular embodiments, the first nucleic acid hybridizes to all or a portion of a first chromosomal band (e.g., it comprises a band-specific probe), and the second nucleic acid hybridizes to all of a portion of a second chromosomal band. Thus, this method utilizes the boolean principles described herein to provide for the detection of chromosomal rearrangements within a single chromosome. Optionally, a population of first nucleic acids can be used, wherein the population comprises the sequences of a plurality of portions of the chromosome. Similarly, a population of second nucleic acids can be used (e.g., a BAC library). Such first nucleic acids may be attached to a plurality of subsections of the substrate, with each subsection having attached thereto a distinct first nucleic acid of the population of first nucleic acids.

More generally, the invention provides methods for comparing nucleic acids, e.g., for comparing a test nucleic acid with a reference nucleic acid. Such methods can be used, for example, for comparing nucleic acids derived from different genera and/or species (e.g., human vs. chimpanzee, Salmonella typhimurium vs. Escherichia coli, etc.), or derived from a single species (e.g., normal vs. abnormal cells, or comparing different strains). Such methods can be used, for example, in generating linkage maps. Methods of the invention include providing a substrate having attached thereto a first nucleic acid comprising the sequence of a first portion of a chromosome or other nucleic acid molecule; contacting the substrate with a test nucleic acid under conditions that permit a first portion of the test nucleic acid to hybridize to all or a portion of said first nucleic acid; contacting the test nucleic with a second nucleic acid under conditions that permit hybridization of all or a portion of the second nucleic acid to a second portion of the test nucleic acid, wherein the second nucleic acid comprises the sequence of (i) a second portion of said chromosome or said other nucleic acid molecule or (ii) a portion of a second chromosome or a second nucleic acid molecule; detecting hybridization of said test nucleic acid to each of the first nucleic acid and the second nucleic acid; and comparing the ability of the test nucleic acid to hybridize to each of the first nucleic acid and the second nucleic acid with the ability of the reference nucleic acid to hybridize to each of the first nucleic acid and the second nucleic acid.

As used herein, the term “chromosomal rearrangement” refers to the relocation of a first portion of a chromosome such that it is adjacent to a second portion of the same or a different chromosome to which it is not normally adjacent, e.g., as the result of a chromosomal translocation, chromosomal deletion, chromosomal insertion, chromosomal inversion, or chromosomal fusion.

As used herein, the term “chromosomal translocation” denotes the physical relocation of a segment of one chromosome to a position on a different chromosome. Conventional nomenclature is used herein to describe the translocation. For example, the occurrence of a translocation between chromosomes 14 and 18 is designated as “t(14;18)”; similarly, the occurrence of a translocation between chromosomes 8 and 21 is designated as “t(8;21).”

The term “chromosomal inversion” denotes the replacement of a section of a chromosome on the reverse order, e.g., as through its removal, rotation by 180 degrees, and reinsertion into the chromosome. Such an inversion may be pericentric (i.e., including the centromere) or peracentric (not involving the centromere)

The term “chromosomal deletion,” as used herein, denotes the removal of a portion of a chromosome, e.g., at least 40 bp, 50 bp, 100 bp, 200 bp, 500 bp, 1000 bp, 2,000 bp, 5000 bp, 100,000 bp, 500,000 bp, 1 Mbp, 50 Mbp, 100 Mbp, or 140 Mbp.

The term “chromosomal fusion” denotes the covalent joining of all or a portion of at least two chromosomes. For example, two chromosomes can be covalently joined at their telomeres, resulting in a double chromosome. Such a double chromosome can be inherited by offspring and result in trisomy, e.g., trisomy 21.

As used herein, the term “colocalization” denotes that two or more labels are in sufficiently close physical proximity so as to indicate that the two or more labels are contained within two or more nucleic acids that are hybridized to a single test nucleic acid (e.g., as in the trimolecular sandwich described herein), or so as to indicate that the two or more labels are contained within a single nucleic acid (e.g., as in the hybrid PCR products synthesized as described herein). A person of ordinary skill in the art would be able readily to determine whether two or more labels are colocalized by taking into consideration the particular nucleic acid hybridization or isolation methods utilized.

As used herein, the term “primer” denotes a nucleic acid molecule that is capable of hybridizing to a target nucleic acid molecule, and of being extended in a template-dependent manner by a DNA or RNA polymerase. A primer is generally 10 or more nucleotides in length such that it forms a stable hybridization product with the template nucleic acid molecule.

The term “substantially all” is used herein to indicate that at least a majority, and preferably, at least 80, 90, or 95%, of a population of primers is detectably labeled with a label (e.g., biotin or DIG).

As used herein, the term “microarray” refers to a substrate to which two or more biopolymers (e.g., a first nucleic acid molecule) are attached in discreet locations. Further, when the biopolymers are nucleic acid molecules (e.g., a first nucleic acid molecule), substantially all of the nucleic acid molecules attached at each discreet location (1) will contain at least one region of sufficient length such that the nucleic acid molecules attached to the substrate are capable of hybridizing to one or more test nucleic acids, and optionally (2) will be derived from the same larger nucleic acid molecule (e.g., a chromosome) or subportion thereof (e.g., a chromosomal band).

The invention offers several advantages over conventional methods for identifying chromosomal translocations. For example, the methods of the invention are not dependent on the quality of chromosomes obtained at metaphase. Additionally, the methods of the present invention do not require prior knowledge of what the disorder may be in order to select and use PCR primers or probes.

Other features and advantages of the invention will be apparent from the claims and from the detailed description thereof. All patents, patent applications and publications cited herein are incorporated herein by reference.

### BRIEF DESCRIPTION OF THE DRAWINGS/FIGURES

FIG. 1 depicts a schematic representation of an overview of one aspect of the invention. In this schematic, a first nucleic acid molecule is attached to a substrate. The substrate and first nucleic acid molecule are then contacted with a sample, which may or may not contain the test nucleic acid molecule, under conditions which will allow hybridization between the first nucleic acid molecule and the test nucleic acid molecule, when present. The substrate and first nucleic acid molecule are also contacted with a second nucleic acid molecule under conditions which will allow hybridization between the second nucleic acid molecule and the test nucleic acid molecule, when present, if the second nucleic acid molecule and the test nucleic acid molecule share at least one region of sequence complementarity.

FIG. 2 is a flow chart of a method for using principles of Boolean logic to isolate nucleic acids bearing a chromosomal translocation.

FIGS. 3A-3C depict the removal of repetitive sequences. FIG. 3A represents labeled Cot DNA; FIG. 3B depicts immobilization of DIG-labeled Cot DNA onto magnetic beads and fishing out” of chromosome 14 repetitive sequences. FIG. 3C depicts immobilization of biotin labeled Cot DNA onto magnetic beads and “fishing out” of chromosome 18 repetitive sequences.

FIGS. 4A-4C depict the removal of sequences common to both chromosomes. FIG. 4A depicts labeled chromosomal DNA; FIG. 4B depicts immobilization of DIG-labeled chromosome 18 onto magnetic beads and “fishing out” of common sequences from chromosome 14; FIG. 4C depicts immobilization of biotin labeled chromosome 14 onto magnetic beads and “fishing out” of common sequences from chromosome 18.

FIGS. 5A-5D depict the isolation and detection of a DNA fragment bearing a chromosomal translocation using PCR methods as described herein.

FIG. 6 depicts the isolation and detection of a DNA fragment bearing a chromosomal translocation using hybridization methods as described herein.

FIG. 7 depicts a 2-dimensional matrix used for identifying chromosomal translocations. In this example, biotin-labeled chromosomes each are immobilized in their own “column” (↓) of the matrix. The test nucleic acid (e.g., patient's DNA) is then hybridized to all immobilized chromosomes. The DIG-labeled chromosomes are then hybridized each to its own “row” (→). A chromosomal translocation is identified, and a signal is generated when a “trimolecular sandwich” is formed by hybridization of the first and second nucleic acids to a test nucleic acid molecule containing a chromosomal rearrangement (see FIG. 7, insert). The presence of the same chromosome in a row and in a column will also generate a signal and thus serves as a control.

### DETAILED DESCRIPTION OF THE INVENTION

The invention provides, in part, compositions and methods that facilitate the identification and characterization of rearrangements or variations in nucleic acid molecules such as chromosomes. Such rearrangements or variations may be involved, for example, in the etiology of disease, e.g., cancer. Other such rearrangements or variations may be identified, for example, in methods for comparing two or more nucleic acid samples.

Methods and compositions of the invention may be used to detect structural differences (e.g., differences in nucleotide sequence) between two or more (e.g., two, three, four, five, six, etc.) nucleic acid molecules. In most instances, a reference nucleic acid molecule will be used and other nucleic acid molecules will be compared to this reference nucleic acid molecule. One example of a reference nucleic acid molecule is a chromosome.

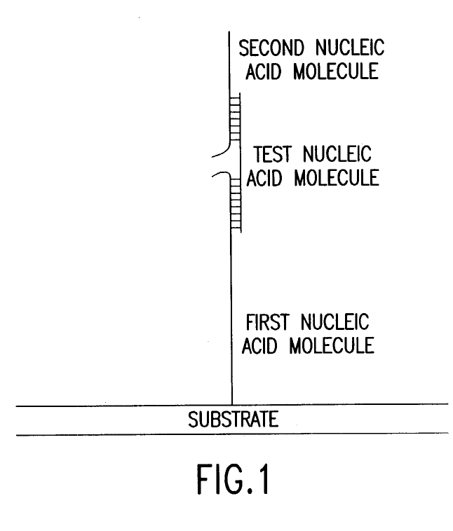

Using the schematic representation set out in FIG. 1 for purposes of illustration, a first nucleic acid molecule corresponding to all or part of a reference nucleic acid molecule may be attached to a substrate and then contacted with a test nucleic acid molecule. In instances where the test nucleic acid molecule shares regions of sequence complementarity with the first nucleic acid molecule attached to the substrate, the conditions will often be such that the two molecule anneal to each other. When the test nucleic acid molecule also shares a region of sufficient sequence complementarity with a second nucleic acid molecule, which will typically have a nucleotide sequence that is different from that of the first nucleic acid molecule, the test nucleic acid molecule may act as a bridge which connects the first nucleic acid molecule to the second nucleic acid molecule. A situation similar to that described above is shown in FIG. 1. As one skilled in the art would recognize, the invention further includes methods similar to those described above but wherein the first nucleic acid molecule is not attached to a substrate and the first nucleic acid molecule and the second nucleic acid molecule act as primers for nucleic acid synthesis. Examples of such variations are described elsewhere herein.

In particular embodiments of the invention, the colocalization of two or more labels can be used to identify test nucleic acid molecules which differ in nucleotide sequence as compared to first nucleic acid molecules. Again using the schematic representation set out in FIG. 1 for purposes of illustration, if the first nucleic acid molecule and second nucleic acid molecule are detectably labeled with different labels, then colocalization of the two different, detectable labels can be used to identify test nucleic acid molecules which share regions of sequence complementarity with both the first nucleic acid molecule and second nucleic acid molecule.

Further, in instances where only the second nucleic acid molecule is detectably labeled, structural differences between the test nucleic acid molecule and the first nucleic acid molecule can be detected by detection of the label at positions on the substrate which correlate with the position of the first nucleic acid molecule. Thus, in such instances, the presence of detectable label can be used to identify variations in nucleic acid structure.

Similarly, when the first nucleic acid molecule and the second nucleic acid molecule are derived from different portions of the same reference nucleic acid molecule, the lack of signal at a particular location on the substrate can be used to detect nucleic acid structures which vary from that of the reference nucleic acid molecule. Thus, for example, it will be possible to form a nucleic acid complex similar to that represented in FIG. 1 when the test nucleic acid molecule has not undergone a structural alteration which would prevent association with the first nucleic acid molecule and the second nucleic acid molecule. Thus, the invention includes methods wherein structural differences between two or more nucleic acid molecules is detected by the lack of the presence of a label or the lack of colocalization of tow or more labels.

Again using the schematic representation set out in FIG. 1 for illustration, the first nucleic acid molecule, the second nucleic acid molecule, and the test nucleic acid molecule, as well as other nucleic acid molecules (e.g., reference nucleic acid molecules) used in methods or present in compositions of the invention, may be derived from any source. As examples, these nucleic acid molecules may be plasmid DNA, chromosomal DNA, mitochondrial DNA, ribosomal RNA, transfer RNA, choloroplast DNA, viral genomic RNA or DNA, etc.

Further, the first nucleic acid molecule, the second nucleic acid molecule, and/or the test nucleic acid molecule, as well as other nucleic acid molecules used in methods of the invention, may each comprise one or more (e.g., one, two, three, four, five, six, etc.) detectable labels. In specific embodiments, different detectable labels may be associated with different nucleic acid molecules (1) used in processes of the invention and (2) present in compositions and/or reaction mixtures of the invention.

Trimolecular Sandwich

As described generally above, a rearrangement in a nucleic acid molecule can be identified by detecting the formation of a trimolecular sandwich. For example, in particular embodiments of the invention, a first nucleic acid (a “capture” probe) including all or a portion of first chromosome or other nucleic acid molecule is attached to a substrate. A portion of a test nucleic acid then is hybridized to a portion of the first nucleic acid (thus hybridizing to a sequence of a portion of a first chromosome or nucleic acid molecule). The test nucleic acid also is contacted with at least one second nucleic acid (a “detection” probe), which comprises the sequence of all or a portion of a second chromosome or other nucleic acid molecule (thus a portion of the test nucleic acid hybridizes to a sequence of a portion of a second chromosome or nucleic acid molecule), thereby forming a trimolecular sandwich. The hybridization of the test nucleic acid to each of the first and second nucleic acids is detected as an indication that the test nucleic acid includes a rearrangement (e.g., a chromosomal translocation).

Nucleic Acids

The first and second nucleic acid molecules each, independently, may include all or a portion of a chromosome or other nucleic acid molecule. Whole chromosomes for use in the invention can be obtained according to conventional methods. For example, whole chromosomes may be obtained by flow sorting (see, e.g., Ferguson-Smith, Eur. J. Human Genet. 5:253-265 (1997); Telenius et al., Genes, Chromosomes, Cancer 3:257-263 (1992); http://www.icnet.uk/axp/facs/davies/chr.html; http://www.cambio.co.uk/star fish/paint/chrompaintpaper.html; and http://www.fastsys.com/images/Xs9& 20.jpg).

As an alternative to using whole chromosomes, portions of chromosomes can be used. For example, a portion of each of the first and/or the second chromosomes, independently, includes about 100 nt to about 300,000 nt (e.g., at least about 200, about 500, about 1000, about 2000, about 3000, about 5,000, about 10,000, about 15,000, about 20,000, about 25,000, about 50,000, about 75,000, about 100,000, about 150,000, about 200,000, about 250,000 nucleotides, etc; or about 100 to 300,000, 100 to 250,000, 100 to 200,000, 100 to 150,000, 100 to 100,000, 100 to 75,000, 100 to 50,000, 100 to 25,000, 100 to 20,000, 100 to 15,000, 100 to 10,000, 100 to 5,000, 100 to 3,000, 100 to 2,000, 100 to 2000, 100 to 1000, 100 to 500, 100 to 200, 200 to 300,000, 200 to 250,000, 200 to 200,000, 200 to 150,000, 200 to 100,000, 200 to 75,000, 200 to 50,000, 200 to 25,000, 200 to 20,000, 200 to 15,000, 200 to 10,000, 200 to 5,000, 200 to 3,000, 200 to 2,000, 200 to 2000, 200 to 1000, 200 to 500, etc). The first and second nucleic acids each should be of a length sufficient to form a stable hybrid molecule with the test nucleic acid. In general, a stable hybrid can be formed between two nucleic acids of at least 20 nucleotides each. Additionally, the sequence of each of the first and second nucleic acids preferably is unique to the particular chromosome from which the nucleic acid is derived, or unique to a particular chromosomal fragment (e.g., a chromosomal band). A portion of a chromosome may be obtained by conventional methods. For example, portions of chromosomes can be obtained by degenerate oligonucleotide primed PCR amplification, using all or a portion of a chromosome as a template for synthesis. Other randomly-primed PCR methods can be used. If desired, a population of chromosome portions can be used, which may contain nucleic acids that are specific to a particular chromosome (e.g., human chromosome 1), or specific to a particular portion of a chromosome.

A variety of materials can be used as the substrate in methods of the invention. The substrate can be any material to which a nucleic acid can be attached and which permits subsequent hybridization of a nucleic acid to the attached nucleic acid. For example, the substrate may be a microchip, a microtiter plate, plastic, a nylon membrane, silicon chips or wafers (e.g., a chip similar to those used in computers), glass (e.g., a microscope slide), a chromatography resin, or beads (e.g., glass or magnetic beads). When the substrate is a biochip, binding of the test nucleic acid to each of the first and second nucleic acids may complete an electrical circuit, thus allowing current to flow, e.g., to send an electrical signal such as to an LCD readout. For example, the first nucleic acid may be attached to gold-coated electrodes on a biochip. The electrodes are electrically insulated to prevent unwanted electrically active molecules in the sample chamber from interfering with the measurements of the test system. When a sample containing the test nucleic acid is introduced into the cartridge, the first nucleic acid(s) on the electrode surface bind to the complementary DNA in the test nucleic acid sample. Electronic labels are attached to the second nucleic acids, which also bind to the test nucleic acid. Binding of the test sequence to both the first nucleic acid and the second nucleic acid connects the electronic labels to the surface, thus altering the bioelectronic circuit on that electrode. When voltage is applied to the sample following hybridization, the labels transfer electrons to the electrode surface, producing an electrical signal, indicating hybridization of the test nucleic acid to each of the first and second nucleic acids.

The substrate can be divided into subsections, if desired, in order to allow multiple assays to be carried out on a single substrate (e.g., multiple assays on a nylon membrane). A microtiter plate, which is already divided into subsections (individual wells) provides a convenient format for carrying out multiple assays. Unique populations of first nucleic acids can be attached to discreet subsections of the substrate. For example, the nucleic acids can be attached to discreet wells of a microtiter plate (e.g., a 96-well plate) or discreet subsections of a microchip, etc. For example, a portion(s) of chromosome 1 may be attached to a first well of a microtiter plate; a portion(s) of chromosome 2 may be attached to a second well; a portion(s) of chromosome 3 may be attached to a third well, etc. Thus, the methods of the invention can readily be carried out in a high throughput format.

Methods for preparing substrates (e.g., microarrays) suitable for use in methods of the invention and in preparing compositions of the invention are known in the art. For example, nucleic acid molecules may be synthesized in situ on the substrate or nucleic acid molecules may be produced and then attached to the substrate.

In situ synthesis methods suitable for use with the present invention include those described in U.S. Pat. No. 6,323,043, the entire disclosure of which is incorporated herein by reference, and PCT publication WO 98/41531. In many instances, in situ synthesis methods involve iterating the sequence by depositing droplets of: (a) a protected monomer onto predetermined locations on a substrate to link with either a suitably activated substrate surface (or with a previously deposited deprotected monomer); (b) deprotecting the deposited monomer so that it can now react with a subsequently deposited protected monomer; and (c) depositing another protected monomer for linking. Different monomers may be deposited at different regions on the substrate during any one iteration so that the different regions of the completed microarray will have different desired biopolymer sequences. One or more intermediate further steps may be required in each iteration, such as oxidation and washing steps. Thus, the invention includes methods which involve in situ synthesis of nucleic acid molecules on substrates, as well as compositions prepared by such methods arid containing compositions containing compositions prepared by such methods. The invention further includes kits which contain microarrays described above, as well as kits which contain nucleic acid molecules and/or instructions for preparing microarrays which can be used in methods of the invention. As one skilled in the art would recognize, methods similar to those described above could be used to synthesize biopolymers on beads and microspheres, which then may be used in methods of the invention or to prepare compositions of the invention.

As noted above, biopolymers (e.g., nucleic acid molecules) may be prepared (e.g., synthesized) first and then contacted with a substrate to produce a substrate to which the biopolymers are connected (e.g., a microarray). Procedures for depositing nucleic acids (e.g., DNA, RNA, DNA-RNA hybrids, RNA or DNA which contains modified bases, etc) on substrates typically involve solubilization of the nucleic acid in a solvent (e.g., an aqueous solvent), followed by loading a small volume of the resulting solution on the tip of a pin or in an open capillary and touching the pin or capillary tube to the surface of the substrate. When the fluid touches the surface, transfer to the substrate occurs of some of the fluid (e.g., about 0.1 nl (nanoliters), about 0.2 nl, about 0.3 nl, about 0.4 nl, about 0.5 nl, about 0.7 nl, about 0.9 nl, about 1.0 nl, about 1.2 nl, about 1.5 nl, about 1.7 nl, about 2.0 nl, about 2.5 nl, about 3.0 nl, about 3.5 nl, about 4.0 nl, about 5.0 nl, about 6.0 nl, about 7.0 nl, about 7.5 nl, about 8.0 nl, about 9.0 nl, about 10.0 nl, about 12.0 nl, about 14.0 nl, about 15.0 nl, about 20.0 nl, about 50.0 nl, about 100 nl, about 500 nl, about 1,000 nl, about 2,000 nl, about 5,000 nl, about 7,000 nl, about 10,000 nl, etc. or from about 0.1 nl to about 10,000 nl, from about 0.1 nl to about 5,000 nl, from about 0.1 nl to about 1,000 nl, from about 0.1 nl to about 100 nl, from about 0.1 nl to about 10.0 nl, from about 0.1 nl to about 5.0 nl, from about 5.0 nl to about 10 nl, from about 0.1 nl to about 15.0 nl, from about 5.0 nl to about 15.0 nl, from about 2.0 nl to about 10 nl, from about 4.0 nl to about 10 nl, from about 5.0 nl to about 12 nl, from about 5.0 nl to about 100 nl, from about 5.0 nl to about 1,000 nl, from about 5.0 nl to about 10,000 nl, from about 100 nl to about 1,000 nl, from about 500 nl to about 2,000 nl, etc.). The pin or capillary tube is typically washed prior to picking up the next type of DNA for spotting onto the substrate. This process is repeated for many multiple different nucleic acid molecules and, eventually, the desired substrate is formed. Alternatively, the biopolymer (e.g., nucleic acid) can be loaded into an inkjet head and sprayed onto the substrate. Such a technique has been described, for example, in PCT publications WO 95/25116 and WO 98/41531, and elsewhere. This inkjet method has the advantage of non-contact deposition. Still other methods for applying biopolymers to substrates include pipetting and positive displacement pumps, such as methods which employ the Bio-Dot A/D3000 Dispenser available from Bio-Dot Inc., Irvine, Calif., USA.

Further, apparati such as those used to prepare dot blots and slot blots may be used to apply biopolymers to substrates. Typically, when such apparati are used, the biopolymers will be applied to the substrates in volumes between 10 microliters and 1.0 milliliter (e.g., about 10 microliters, about 30 microliters, about 50 microliters, about 100 microliters, about 500 microliters, about 1,000 microliters, etc. or from about 10 microliters to about 1,000 microliters, from about 100 microliters to about 1,000 microliters, from about 500 microliters to about 1,000 microliters, from about 10 microliters to about 500 microliters, from about 10 microliters to about 300 microliters, from about 10 microliters to about 100 microliters, from about 100 microliters to about 500 microliters, etc.).

When preparing substrates which contain a plurality of biopolymers, a number of practical factors are typically considered during fabrication. Examples of these factors include the following. First, the sensitivity of methods employing microarrays is dependent on having reproducible spots on the substrate. For example, the specific biopolymer present at each location on the substrate typically may be known and, for most purposes, the spotted area should be uniformly coated with the biopolymer. Second, since biopolymers are typically expensive to produce in purified form, a minimum amount of the solution which contains the biopolymer, and thus a minimum amount of the biopolymer itself, should be loaded into any of the transfer devices used to deposit the biopolymer on the substrate. Third, any cross contamination of different biopolymers should be lower than the sensitivity of the final microarray as used in a particular assay, to prevent false positive signals. Therefore, the transfer device should be easily cleaned after each type of biopolymer is deposited or the device will generally be inexpensive enough to be a disposable. Finally, since the quantity of the assay sample (e.g., the sample which contains the test nucleic acid molecule) is often limited, it is advantageous to make the spots small and closely spaced.

As suggested above, the amount of nucleic acid applied to substrates in each spot will typically be relatively small. For example, the amount of nucleic acid applied in each spot may be about 0.1 picrograms (pg), about 0.2 pg, about 0.4 pg, about 0.5 pg, about 0.7 pg, about 0.9 pg, about 1.0 pg, about 2.0 pg, about 3.0 pg, about 5.0 pg, about 10.0 pg, about 15.0 pg, about 20.0 pg, about 30.0 pg, about 40.0 pg, about 50.0 pg, about 80.0 pg, about 100 pg, about 200 pg, about 300 pg, about 400 pg, about 500 pg, about 800 pg, about 1,000 pg, about 1,500 pg, about 2,000 pg, about 4,000 pg, about 5,000 pg, about 10,000 pg, about 20,000 pg, about 50,000 pg, about 100,000 pg, about 200,000 pg, about 500,000 pg, about 1,000,000 pg, about 2,000,000 pg, about 3,000,000 pg, etc. Further, the amount of nucleic acid applied in each spot may be within the following ranges: from about 0.1 pg to about 5,000 pg, from about 5.0 pg to about 5,000 pg, from about 50.0 pg to about 5,000 pg, from about 100 pg to about 5,000 pg, from about 0.1 pg to about 2,000 pg, from about 0.1 pg to about 1,000 pg, from about 0.1 pg to about 500 pg, from about 0.1 pg to about 200 pg, from about 10.0 pg to about 1,000 pg, from about 10.0 pg to about 500 pg, from about 10.0 pg to about 100 pg, from about 40.0 pg to about 5,000 pg, from about 40.0 pg to about 1,000 pg, from about 40.0 pg to about 500 pg, from about 40.0 pg to about 200 pg, from about 50.0 pg to about 5,000 pg, from about 50.0 pg to about 1,000 pg, from about 50.0 pg to about 500 pg, from about 50.0 pg to about 200 pg, from about 100 pg to about 1,000 pg, from about 100 pg to about 500 pg, from about 200 pg to about 5,000 pg, from about 200 pg to about 1,000 pg, from about 200 pg to about 500 pg, from about 400 pg to about 5,000 pg, from about 400 pg to about 1,000 pg, from about 400 pg to about 500 pg, from about 100 pg to about 3,000,000 pg, from about 100,000 pg to about 3,000,000 pg, from about 500,000 pg to about 3,000,000 pg, from about 1,000,000 pg to about 3,000,000 pg, from about 10,000 pg to about 100,000 pg, from about 50,000 pg to about 200,000 pg, etc.

Further, the spots themselves may be of any shape, but will typically be roughly round, oval, square, or rectangular.

Also, the spots will typically be relatively small in size because, as noted above, small spots may be positioned closer together than large spots to generate microarrays of higher density. Individual spots of microarrays of the invention will typically cover an area on the substrate of about 0.01 mm², about 0.02 mm², about 0.03 mm², about 0.04 mm², about 0.05 mm², about 0.06 mm², about 0.07 mm², about 0.08 mm², about 0.09 mm², about 0.1 mm², about 0.2 mm², about 0.3 mm², about 0.4 mm², about 0.5 mm², about 0.6 mm², about 0.8 mm², about 1.0 mm², about 1.5 mm², about 2.0 mm², about 4.0 mm², about 5.0 mm², about 7.0 mm², about 8.0 mm², about 10.0 mm², about 12.0 mm², about 15.0 mm², about 20.0 mm², about 25.0 mm², about 30.0 mm², about 40.0 mm², about 50.0 mm², about 60.0 mm²,about 70.0 mm², about 80.0 mm², about 100 mm², etc. Individual spots of microarrays of the invention may also cover areas on the substrate of from about 0.01 mm² to about 100 mm², from about 0.1 mm² to about 100 mm², from about 1.0 mm² to about 100 mm², from about 5.0 mm² to about 100 mm², from about 10.0 mm² to about 100 mm², from about 20.0 mm² to about 100 mm², from about 40.0 mm² to about 100 mm², from about 50.0 mm² to about 100 mm², from about 0.01 mm² to about 80.0 mm², from about 0.01 mm² to about 70.0 mm², from about 0.01 mm² to about 50.0 mm², from about 0.01 mm² to about 40.0 mm², from about 0.01 mm² to about 30.0 mm², from about 0.01 mm² to about 20.0 mm², from about 0.01 mm² to about 10.0 mm², from about 0.01 mm² to about 5.0 mm², from about 0.01 mm² to about 3.0 mm², from about 0.01 mm² to about 2.0 mm², from about 0.01 mm² to about 1.0 mm², from about 1.0 mm² to about 50 mm², from about 1.0 mm² to about 30 mm², from about 1.0 mm² to about 20 mm², from about 1.0 mm² to about 10 mm², from about 3.0 mm² to about 50 mm², from about 3.0 mm² to about 20 mm², from about 3.0 mm² to about 10 mm², from about 5.0 mm² to about 50 mm², from about 5.0 mm² to about 30 mm², from about 5.0 mm² to about 20 mm², from about 5.0 mm² to about 10 mm², etc.

In addition, microarrays suitable for use with the invention include those which contain a considerable number of spots which contain different biopolymers. While the biopolymers present in a number of these spots may be identical, many of the spots (e.g., more than 70%, 75%, 80%, 85%, 90%, 95%, 98%, 99%, 99.5% of the total spots present) will contain biopolymers which are different. Spots with identical biopolymers may function, for example, as control spots. In certain instances, the individual biopolymer molecules in each spot, or present on beads or microspheres used in methods of the invention, will substantially all be identical or will at least share a region of significant size (e.g., a region larger than 70%, 75%, 80%, 85%, 90%, 95%, 98%, 99%, 99.5% of the average size of the biopolymers molecules present) which is identical. In other instances, the individual biopolymer molecules in each spot, or present on beads or microspheres used in methods of the invention, will be nucleic acid molecules derived from the same nucleic acid molecule (e.g., the same chromosome) or the same region of a nucleic acid molecule (e.g., the same band of a chromosome). Further, libraries of nucleic acid molecules derived from the same nucleic acid molecule or the same region of a nucleic acid molecule may be present in each spot. These libraries may be present in vectors such as, for example, lambda phage, BAC (bacterial artificial chromosome), YAC (yeast artificial chromosome) or MAC (mammalian artificial chromosome) vectors.

The average size of nucleic acids derived from the same nucleic acid molecule or the same region of a nucleic acid molecule and present in each spot may vary considerably. Examples of such sizes include about 1,000 base pairs, about 5,000 base pairs, about 10,000 base pairs, about 100,000 base pairs, about 500,000 base pairs, about 1,000,000 base pairs, about 2,000,000 base pairs, about 5,000,000 base pairs, about 10,000,000 base pairs, about 50,000,000 base pairs, about 100,000,000 base pairs, about 150,000,000 base pairs, etc. or from about 1,000 base pairs to about 150,000,000 base pairs, from about 20,000 base pairs to about 150,000,000 base pairs, from about 50,000 base pairs to about 150,000,000 base pairs, from about 200,000 base pairs to about 150,000,000 base pairs, from about 1,000,000 base pairs to about 150,000,000 base pairs, from about 50,0000,000 base pairs to about 150,000,000 base pairs, from about 50,000 base pairs to about 150,000 base pairs, from about 50,000 base pairs to about 500,000 base pairs, from about 50,000 base pairs to about 5,000,000 base pairs, etc. Nucleic acid molecules used as the test nucleic acid or second nucleic acid may also be of average sizes described above.

Examples of the number of spots which may be present on microarrays of the invention, or used in methods of the invention, include about 2, about 5, about 10, about 20, about 30, about 50, about 70, about 90, about 100, about 150, about 200, about 400, about 500, about 600, about 800, about 1,000, about 1,200, about 1,500, about 1,700, about 1,900, about 2,000, about 2,300, about 2,500, about 2,700, about 2,900, about 3,000, about 3,500, about 4,000, about 5,000, about 6,000, about 7,000, about 8,000, about 10,000, about 15,000, about 20,000, about 25,000, about 30,000 about 35,000, about 40,000, about 45,000, about 50,000, about 60,000, about 80,000, about 100,000, about 150,000, about 200,000, etc. Examples of ranges of numbers of spots which may be present on microarrays of the invention, or used in methods of the invention, include from about 2 to about 60,000, from about 2 to about 50,000, from about 2 to about 40,000, from about 2 to about 30,000, from about 2 to about 20,000, from about 2 to about 10,000, from about 2 to about 5,000, from about 2 to about 3,000, from about 2 to about 2,000, from about 2 to about 1,000, from about 2 to about 500, from about 2 to about 300, from about 2 to about 200, from about 2 to about 100, from about 2 to about 50, from about 2 to about 20, from about 10 to about 60,000, from about 50 to about 60,000, from about 100 to about 60,000, from about 500 to about 60,000, from about 800 to about 60,000, from about 1,000 to about 60,000, from about 2,000 to about 60,000, from about 5,000 to about 60,000, from about 7,000 to about 60,000, from about 10,000 to about 60,000, from about 15,000 to about 60,000, from about 20,000 to about 60,000, from about 30,000 to about 60,000, from about 40,000 to about 60,000, from about 20 to about 50,000, from about 20 to about 40,000, from about 20 to about 20,000, from about 20 to about 10,000, from about 20 to about 5,000, from about 20 to about 1,000, from about 20 to about 500, from about 20 to about 200, from about 100 to about 50,000, from about 100 to about 40,000, from about 100 to about 30,000, from about 100 to about 20,000, from about 100 to about 10,000, from about 100 to about 5,000, from about 100 to about 1,000, from about 100 to about 500, from about 500 to about 50,000, from about 500 to about 40,000, from about 500 to about 30,000, from about 500 to about 20,000, from about 500 to about 10,000, from about 500 to about 5,000, from about 500 to about 1,000, from about 500 to about 800, from about 5,000 to about 100,000, from about 10,000 to about 200,000, from about 20,000 to about 150,000, etc.

The density of spots present on microarrays of the invention, or used in methods of the invention, may vary greatly. Examples of such spot densities include about 1 spot/cm², about 2 spots/cm², about 3 spots/cm², about 4 spots/cm², about 5 spots/cm², about 10 spots/cm², about 15 spots/cm², about 20 spots/cm², about 25 spots/cm², about 30 spots/cm², about 35 spots/cm², about 40 spots/cm², about 45 spots/cm², about 50 spots/cm², about 55 spots/cm², about 60 spots/cm², about 65 spots/cm², about 70 spots/cm², about 75 spots/cm², about 80 spots/cm², about 85 spots/cm², about 90 spots/cm², about 95 spots/cm², about 100 spots/cm², about 110 spots/cm², about 120 spots/cm², about 130 spots/cm², about 140 spots/cm², about 150 spots/cm², about 200 spots/cm², about 300 spots/cm², about 400 spots/cm², about 500 spots/cm², about 600 spots/cm², about 700 spots/cm², about 800 spots/cm², about 900 spots/cm², about 1,000 spots/cm², etc. Examples of ranges of the density of spots present on microarrays of the invention, or used in methods of the invention, include from about 5 spots/cm² to about 1,000 spots/cm², from about 10 spots/cm² to about 1,000 spots/cm², from about 20 spots/cm² to about 1,000 spots/cm², from about 40 spots/cm² to about 1,000 spots/cm², from about 50 spots/cm² to about 1,000 spots/cm², from about 60 spots/cm² to about 1,000 spots/cm², from about 70 spots/cm² to about 1,000 spots/cm², from about 80 spots/cm² to about 1,000 spots/cm², from about 100 spots/cm² to about 1,000 spots/cm², from about 150 spots/cm² to about 1,000 spots/cm², from about 200 spots/cm² to about 1,000 spots/cm², from about 250 spots/cm² to about 1,000 spots/cm², from about 300 spots/cm² to about 1,000 spots/cm², from about 400 spots/cm² to about 1,000 spots/cm², from about 500 spots/cm² to about 1,000 spots/cm², from about 700 spots/cm² to about 1,000 spots/cm², from about 30 spots/cm² to about 1,000 spots/cm², from about 30 spots/cm² to about 800 spots/cm², from about 30 spots/cm² to about 600 spots/cm², from about 30 spots/cm² to about 500 spots/cm², from about 30 spots/cm² to about 400 spots/cm², from about 30 spots/cm² to about 200 spots/cm², from about 30 spots/cm² to about 100 spots/cm², from about 30 spots/cm² to about 70 spots/cm², from about 50 spots/cm² to about 800 spots/cm², from about 50 spots/cm² to about 400 spots/cm², from about 50 spots/cm² to about 200 spots/cm², from about 50 spots/cm² to about 100 spots/cm², from about 100 spots/cm² to about 800 spots/cm², from about 100 spots/cm² to about 400 spots/cm², from about 100 spots/cm² to about 200 spots/cm², from about 100 spots/cm² to about 150 spots/cm², etc.

Microarrays suitable for use with the present invention may be prepared manually, e.g., using a pipette, or may be prepared using a computer directed robotic device(s). Methods for preparing microarrays using such devices are described, for example, in U.S. Pat. No. 5,807,522, the entire disclosure of which is incorporated herein by reference. For example, a specially designed mechanical robot, which produces a probe spot on the substrate by dipping a pin head into a fluid containing an off-line synthesized DNA and then spotting it onto the slide at a predetermined position may be used to generate microarrays. Washing and drying of the pins are typically required prior to the spotting of a different probe on the substrate. Further, the spotting pin, and/or the stage carrying the substrate(s) may move along the X, Y and Z axes in coordination to deposit samples at controlled positions on the substrate(s). Similar computer directed robotic devices can be used to prepare beads and microspheres which contain biopolymers. For example, robotic devices can be used to contact beads or microspheres present in wells with solutions which contain biopolymers.

Biopolymers may attach to the substrate by any number of physical or chemical ways. Further, the biopolymers may be permanently or reversibly attached to the substrate. For example, biopolymer molecules may be attached to substrates by covalent bonds. For instance, nucleic acid molecules with a terminal amine group may be covalently linked to a substrate which contains aldehyde groups free for reaction.

Substrates with any number of reactive groups for the attachment of biopolymers (e.g., nucleic acid molecules) may be used to prepare microarrays, or other solid supports, for use in the present invention. A wide variety of such reactive groups are known in the art. Examples of substrates to which biopolymers such as nucleic acid molecules can be attached include substrates which are silynated. Additional examples of suitable substrates include those which contain aldehyde groups or are coated with poly-lysine (e.g., poly-L-lysine). Nucleic acid molecules which are attached to substrates may be modified (e.g., contain an added chemical group which reacts to form covalent or non-covalent bonds with the substrate or chemical groups on the surface thereof) or unmodified nucleic acids. Examples of processes for adding reactive groups to substrates and for the attachment of biopolymers to such modified substrates are described in U.S. Pat. No. 6,048,695, the entire disclosure of which is incorporated herein by reference.

Substrates suitable for use in preparing microarrays for use in the present invention may be obtained, for example, from Genetix Ltd. (St. James, N.Y. 11780, USA, 800-385-9248). For example, the products sold by Genetix Ltd. as catalog number K2625 are aldehyde slides for covalently binding biopolymers via Schiff base aldehyde-amine chemistry. These slides are suitable for preparing nucleic acid microarrays by the spotting of, for example, either amino-modified PCR products or unmodified PCR products, as well as microarrays prepared by the spotting of other biopolymers which contain suitable reactive groups (e.g., amine groups).

Substrates suitable for use in methods and compositions of the invention include beads and microspheres. Using the format set out in FIG. 1 for illustration, nucleic acid molecules corresponding to different reference nucleic acids may be connected to beads or microspheres so that individual beads or microspheres comprise nucleic acid molecules which are either identical in sequence or share regions of identical sequence. Beads or microspheres which comprise different nucleic acids may then be mixed and contacted with a test nucleic acid and a second nucleic acid molecule. Thus, the beads or microspheres act as the substrate in this instance. Further, these beads or microspheres may be detectably labeled to identify beads or microspheres which contain specific first nucleic acid molecules. Methods for using beads and microspheres in processes such as those described above are set out in PCT Publication WO 00/71243, the entire disclosure of which is incorporated herein by reference.

The first nucleic acid can be attached to the substrate using any conventional method. For example, a solution containing the first nucleic acid(s) can be applied to the substrate, and attached to the substrate by allowing the solution to dry onto the substrate, and then by treating the nucleic acid with ultraviolet light (e.g., at 320 nm for 45 seconds). If desired, the nucleic acid can be attached to the substrate using a first moiety, i.e., a “linker” moiety, which may also serve as a detectable label. For example, the first nucleic acid may be detectably labeled with DIG, and the DIG moiety may also be used to attach the nucleic acid to the substrate. Thus, in such methods, the substrate may have attached thereto a second moiety, i.e., an “anchor” moiety, that can bind to the linker moiety and thus anchor the nucleic acid to the substrate. If the linker moiety is biotin, the anchor moiety may be, for example, streptavidin, or vice versa. Other combinations of linker moieties and anchor moieties are known in the art and can be used in the invention, e.g., biotin and avidin, amine and aldehyde groups, etc.

The test nucleic acid may be derived from any source, e.g., an organism such as a single-cell or multicellular eukaryote. Typically, the test nucleic acid includes at least about 40 nucleotides, and may be as large as a whole chromosome, e.g., about 256,000,000 nt; in particular embodiments, the test nucleic acid includes about 200 to about 250,000 nucleotides, about 200 to about 200,000 nucleotides, about 200 to about 100,000 nucleotides, about 200 to about 75,000 nucleotides, about 200 to about 50,000 nucleotides, about 200 to about 40,000, about 200 to about 30,000 nucleotides, about 200 to about 20,000 nucleotides, about 200 to about 10,000 nucleotides, about 200 to about 5000 nucleotides, about 200 to about 4,000, about 200 to about 3,000 nucleotides, about 200 to about 2,000 nucleotides, about 200 to about 1,000 nucleotides, about 200 to about 500 nucleotides. The length of the test nucleic acid can be adjusted using conventional methods, e.g., by digestion with restriction enzymes. In particular embodiments, the test nucleic acid is suspected of containing a chromosomal rearrangement relative to a reference nucleic acid (e.g., patient DNA vs. DNA of a healthy subject). As described herein, the test nucleic acid can be derived from a nucleic acid sample which is to be compared to a reference sample. For example, nucleic acids of the genomes of two or more organisms can be compared, e.g., wild-type vs. mutant; a strain of interest vs. a reference strain; a species of interest vs. a reference species, etc).

The test nucleic acid may be hybridized to the first nucleic acid (the “capture” probe), preferably while the first nucleic acid is attached to the substrate. Hybridizations are performed under conditions that permit a portion of the test nucleic acid to hybridized to the first or second nucleic acid. Conventional hybridization conditions can be used. For example, stringent hybridization conditions can be used. Such conditions are comparable to an overnight incubation at 42° C. in a solution comprising 50% formamide, 5×SSC (750 mM NaCl, 75 mM sodium citrate), 50 mM sodium phosphate (pH 7.6), 5× Denhardt's solution, 10% dextran sulfate, and 20 μg/ml denatured, sheared salmon sperm DNA, followed by washing in 0.1×SSC at about 65° C. “Low stringency conditions” are comparable to an overnight incubation at 37° C. in a solution comprising 6×SSPE (20×SSPE=3M NaCl; 0.2M NaH₂PO₄; 0.02M EDTA, pH 7.4), 0.5% SDS, 30% formamide, 100 μg/ml salmon sperm blocking DNA; followed by washes at 50° C. with 1×SSPE, 0.1% SDS. Low stringency conditions may be preferred if the hybridizing nucleic acids are not expected to be exactly complementary.

The second nucleic acid used in the formation of the trimolecular sandwich may be hybridized to a portion of the test nucleic acid. Typically, the second nucleic acid is hybridized to the test nucleic acid after the test nucleic acid is hybridized to the first nucleic acid. Optionally, hybridization of the second nucleic acid to the test nucleic acid can be carried out before, or simultaneously with, hybridization of the test nucleic acid to the first nucleic acid.

The second nucleic acid will typically comprise a nucleic acid sequence that is distinct from the first nucleic acid. When methods of the invention are used to identify chromosomal translocations, the second nucleic acid will typically comprise a distinct nucleic acid from a chromosome that is distinct from the chromosome from which the first nucleic acid is derived. In other embodiments, as described infra, the first and second nucleic acids may comprise distinct sequences of a single chromosome, e.g., chromosomal band-specific nucleic acids, thus permitting the identification of chromosomal rearrangements within a single chromosome (e.g., deletions, inversions, or fusions). The second nucleic acid can be hybridized to the test nucleic acid under conditions such as those described herein for hybridization of the test nucleic acid to the first nucleic acid.

When the first and second nucleic acids used as “capture” and “detection” probes, respectively, are derived from the same chromosome, the length of the test nucleic acid preferably will generally be sufficiently short such that the selected probes would not both be expected to hybridize to the test nucleic acid except as the result of a chromosomal rearrangement. For example, if the selected first and second nucleic acids are known normally to be separated on a chromosome by 5000 or more base pairs, the population of test nucleic acids generally will be prepared such that the size of the test nucleic acids generally will be 5000 bp or less. Thus a test nucleic acid would not be expected to hybridize to both the first and second nucleic acids unless the test nucleic acid contains a chromosomal rearrangement. If desired, the size of the test nucleic acid in a sample can be adjusted by treating the sample of nucleic acids with a restriction enzyme(s). For example, treatment of the test nucleic acid with a restriction enzyme having a hexanucleotide target would be predicted to digest the test nucleic acid once every 4⁶ (4096) base pairs. On a normal human chromosome 2, band 2p25 lies more than 4096 base pairs away from band 2p11.2. Thus, a test nucleic acid that has been treated with a restriction enzyme having a hexanucleotide target would be expected to be sufficiently short so as not to contain sequences corresponding to the first and second nucleic acids except as the result of a chromosomal rearrangement. In an alternative method, the size of the test nucleic acid can be adjusted by shearing the nucleic acid and, optionally, removing large nucleic acids.

Preferably, repetitive sequences that may be contained in the first and/or second nucleic acids are blocked or removed prior to contacting the first and/or second nucleic acids with the test nucleic acid. For example, Cot 1 DNA (Roche) can be immobilized on magnetic beads (Roche) and then used to extract repetitive sequences in a solution of nucleic acids comprising portions of chromosomes as the first or second nucleic acids, which can be accomplished using conventional techniques. Alternatively, DNA such as Cot1 DNA, salmon sperm DNA or herring sperm DNA can be hybridized to the first and/or second nucleic acids to block repetitive sequences in accordance with conventional techniques.

The first and/or second nucleic acids can be detectably labeled, if desired. Any moiety can be used as the label, provided that the presence or absence of the label can be detected. Suitable labels include, for example, enzymatic labels, fluorescent labels, radioisotopic labels (e.g., P³²), chemiluminescent labels, and antigenic labels. Preferred labels include, without limitation, digoxigenin (DIG), biotin, and digoxin. Examples of suitable enzyme labels include alkaline phosphatase, acetylcholine esterase, alpha-glycerol phosphate dehydrogenase, alkaline phosphatase, asparaginase, β-galactosidase, catalase, delta-5-steroid isomerase, glucose oxidase, glucose-6-phosphate dehydrogenase, glucoamylase, glycoamylase, luciferase, malate dehydrogenase, peroxidase, staphylococcal nuclease, triose phosphate isomerase, urease, and yeast-alcohol dehydrogenase. Examples of suitable fluorescent labels include a fluorescein label, an isothiocyanate label, a rhodamine label, a phycoerythrin label, a phycocyanin label, an allophycocyanin label, an o-phthaldehyde label, a fluorescamine label, etc. Examples of suitable chemiluminescent labels include a luminal label, an isoluminal label, an aromatic acridinium ester label, an imidazole label, an acridinium salt label, an oxalate ester label, a luciferin label, and an aequorin label, etc. Conventional methods can be used to attach such labels to the first and/or second nucleic acids. Similarly, conventional detection methods can be used to identify nucleic acids having such labels attached thereto.

Detectably labeling the first and/or second nucleic acids with first and second labels, respectively, can facilitate the identification of a test nucleic acid that hybridizes to each of the first and the second nucleic acids and which contains a chromosomal rearrangement or other structural variation. For example, the detection of the colocalization of the first and second labels is an indication that the test nucleic acid hybridized to each of the first and second nucleic acids and thus contains the breakpoint of a chromosomal translocation or other structural variation. Colocalization of the detectable labels can be identified in accordance with standard techniques. For example, the first nucleic acid can be attached to a discreet subsection of the substrate, e.g., a well of a microtiter plate, a portion of a nylon membrane, a portion of a glass substrate, a portion of a microchip, etc. The test nucleic acid then is hybridized to the first nucleic acid, and the second nucleic acid is hybridized to the test nucleic acid. Any second nucleic acid that is not hybridized to the test nucleic acid is removed (e.g., by washing it away). The colocalization of the detectable labels can be identified by detecting both labels in a single subsection of the substrate, e.g., in a single well of a microtiter plate, or in a single portion of a nylon membrane, glass substrate, or microchip, etc. Conventional methods for detecting labels can be used, e.g., fluorescence detection methods, radioisotopic detection methods, enzymatic detection methods, chemiluminescent detection methods, etc. As an alternative to detecting labels on each of the first and second nucleic acids, the presence of the second nucleic acid can be detected in a sample that is thought to contain the first nucleic acid, without the need to detect a label on the first nucleic acid and without the need even to label the first nucleic acid. For example, the first nucleic acid may be attached to a particular subsection of a substrate (e.g., a particular well of a microtiter plate); the test nucleic acid then is hybridized to the first nucleic acid; excess, non-hybridized test nucleic acid may be removed; and the second nucleic acid is hybridized to the test nucleic acid; and excess, non-hybridized second nucleic acid is removed. Following the removal of any non-hybridized second nucleic acid, the detection of a second nucleic acid in the same particular subsection of the substrate (e.g., in the same well of a microtiter plate) is an indication that the test nucleic acid hybridized to each of the first the second nucleic acids, since that particular subsection is known also to contain the first nucleic acid. If the second nucleic acid is detectably labeled, the presence of the second label on the second nucleic acid can be detected using conventional techniques, as indication that the test nucleic acid hybridized to both the first and second nucleic acids. In yet other particular embodiments, the presence of the second nucleic acid can be detected by eluting the second nucleic acid from the sample (e.g., by increasing the temperature of the sample to destabilize the hybridization of the second nucleic acid to the test nucleic acid), and by cloning and/or sequencing the second nucleic acid in accordance with conventional techniques, as an indication that the test nucleic acid is hybridized to both the first and second nucleic acids.

Band-Specific Probes

If desired, the first and second nucleic acids each can include a portion of a chromosome, and can be derived from the same chromosome. The use of portions of a single chromosome facilitates the identification of chromosomal rearrangements involving a single chromosome, e.g., deletions, inversions, or fusions of a single chromosome. If desired, the first and/or second nucleic acids may include a sequence that is unique to a particular chromosomal band (i.e., band-specific nucleic acid). Examples of band-specific nucleic acids include, without limitation, nucleic acids that specifically hybridize to the following portions of human chromosomes: 1pT, 1p36.3, 1p31, 1p22, 1p11-12, 1q11-12, 1q21, 1q24, 1q32, 1q41, 1q43, 1q44, 1qT, 2pT, 2p25, 2p24, 2p23, 2p22, 2p21, 2p16, 2p13-15, 2p12, 2p11.2, 2P11Q11, 2q12, 2q13-14.2, 2q14.321, 2q22, 2q23, 2q24, 2q31-32.1, 2q32.2-32.3, 2q33, 2q34, 2q35, 2q36, 2q37.1-2, 2q37.3,qT, 3pT, 3p26, 3p25, 3p24, 3p22-23, 3p21, 3p14, 3p13, 3p12, 3p11, 3q12, 3q13, 3q21, 3q22-23, 3q24, 3q25, 3q26, 3q27, 3q28-29, 3qT, 4pT, 4p16.2-3, 4p14, 4p13, 4p11, 4q11-12, 4q21.121-2, 4q21.3, 4q22, 4q23, 4q24-25, 4q26, 4q28, 4q31, 4q34, 4q35, 4qT, 5pT, 5p15, 5p14, 5p13, 5p11-12, 5pcen, 5q11, 5q12, 5q13, 5q14-15, 5q21, 5q31, 5q32, 5q33, 5q34, 5q35, 5q35.2-3, 5qT, 6pT, 6p25, 6p23-24, 6p21.3, 6q11-12, 6q13-14, 6q14-15 6q16-21, 6q22a, 6q2-223, 6q23-24, 6q25, 6q27, 6qT, 7pT, 7p22, 7p21, 7p14, 7p12-13, 7q11.2, 7q21, 7q31, 7q32-34, 7q35, 7q36, 7qT, 8P231233, 8p22, 8p21, 8p11.2, 8q12-13, 8q21.1, 8q22, 8q23, 8q24.1-24.2, 8q24.3, 8qT, 9pT, 9p22-23, 9p21, 9p13, 9q12-13, 9q22, 9q31, 9q34, 9qT, 10pT, 10 p15, 10p13-15, 10p12, 10q21, 10q24-25, 10q26, 10qT, 11pT, 11P1545, 11P1513, 11p14.1, 11P11213, 11q11-12, 11Q1312, 11q14, 11q21-22, 11q23-24, 11q24-25, 11qT, 12pT, 12p13.3, 12p13, 12p12, 12q13, 12q21, 12q22-23, 12Q2413, 12Q2445, 12qT, 13q12, 13q14, 13q21, 13q22, 13q31, 13q32, 13q33, 13q34, 13qT, 14q24-31, 14q32, 14qT, 15q1213, 15q14-15, 15q21, 15q22-24, 15q25, 5q26.1, 15Q2623, 15qT, 16pT, 16p13.3, 16p12, 16p11.2, 16q21, 16q22-23, 16q24, 16qT, 17pT, 17P1323, 17p13, 17p11, 17q21, 17q22-24, 17q25, 17qT, 18pT, 18p13, 18P1112, 18q11, 18q12, 18q21, 18q23,18qT, 19pT, 19p13.3, 19p13.2, 19q13.1, 19q13.2, 19q13.3, 19q13.4, 19qT, 20pT, 20p13, 20p12, 20p11, 20q11.2, 20Q12131, 20q13.2, 20Q1323, 20qT, 21q1, 21q2, 22q13.3, 22qT, XpT, Xp22.3, Xp22.1.-2, Xp21, Xp11.2-11.4, Xp11.1, Xq12-13, Xq21, Xq23-24, Xq27-28, XqT, YpT, and YqT. Numerous band-specific nucleic acids are known in the art and can be used in the invention (see, e.g., band-specific probes described by Invitrogen Corp. (Carlsbad, Calif.), Cambio Ltd. (Cambridge, England), Vysis, Inc. (Downers Grove, Ill.), Qbiogene (Carlsbad, Calif.), and A. L. Tech Biomedical, Inc. (Rockville, Md.); see also Meltzer et al., Nature Genetics 1:24-28 (1992), Guan et al., Hum. Mol. Genet. 2:1117-1121 (1993), Guan et al., Genomics 22:101-107 (1994), Meltzer et al., Cancer Genet. Cytogenet. 93:29-32 (1997), Chen et al., Am. J Med. Genet. 71:160-166), and Jonveaux, Ann. Genet. 38:5-12 (1995)). Of course, portions of chromosomes derived from organisms other than humans can be used in methods and compositions of the invention. Examples of such organisms include non-human primates (e.g., chimpanzees, monkeys, gorillas, etc.), insects, worms, (e.g., C. elegans, etc), yeast (e.g., S. cerevisiae, S. pombe, etc.), and protozoans.

Thus, the methods described herein for identifying chromosomal translocations can also be utilized with band-specific nucleic acids serving as the first and second nucleic acids. To identify a chromosomal deletion, the first and second nucleic acids are derived from the same chromosome, e.g., chromosome 2. For example, the first nucleic acid (i.e., the “capture” probe) may include sequences specific to chromosome band 2p25, whereas the second nucleic acid (i.e., the “detection” probe) may contain sequences specific to chromosome band 2p11.2. The test nucleic acid that is used is sufficiently short (e.g., by treatment of a sample of DNA with restriction enzymes) such that sequences corresponding both to band 2p25 and to band 2p11.2 would not be expected to be contained within the test nucleic acid except as the result of a chromosomal rearrangement.

If desired, test nucleic acid identified as containing a chromosomal rearrangement or other alteration can subsequently be sequenced or mapped by conventional methods to confirm the presence and identity of the chromosomal rearrangement or other alteration. Having selected the first and second nucleic acids (e.g., band-specific nucleic acids derived from a single chromosome) and having prepared the test nucleic acid, the hybridization method for identifying chromosomal rearrangements or nucleic acid alterations can be carried out as described herein. The identification of a test nucleic acid that hybridizes both to the first and to the second nucleic acids typically is an indication that the test nucleic acid contains a chromosomal deletion, resulting in two, normally more distant, sequences being brought closer together.

In related embodiments, the invention also provides methods for detecting insertions, e.g., by identifying a test nucleic acid that hybridizes to one, but not both, of a first and a second nucleic acid. The first and second nucleic acids chosen for use in this method should be sufficiently close together on a normal chromosome or reference nucleic acid molecule such that the test nucleic acid would be predicted to hybridize to both the first and second nucleic acids except in the event of a rearrangement. For example, nucleic acids corresponding to human bands 19p13.3 and 19p13.2 can be used as the first and second nucleic acids. The test nucleic acid should be of a sufficient length such that it would be predicted to hybridize to both of the first and second nucleic acids, except in the event of a rearrangement. Thus, the hybridization methods described herein can be carried out using as the capture and detection probes two portions of a chromosome or other nucleic acid molecule which normally are close together, e.g., on a chromosome. A test nucleic acid that hybridizes to one, but not to both, of the capture and detection probes is identified as containing an insertion. If desired, the ability of the test nucleic acid to hybridize to the first and second nucleic acids can be compared with the ability of a reference nucleic acid (e.g., a “normal” nucleic acid) to hybridize to the first and second nucleic acids. For example, the reference nucleic acid may be derived from a normal, healthy subject, whereas the test nucleic acid may be derived from a patient suspected of carrying an insertion. If desired, a test nucleic acid identified as having a rearrangement can subsequently be sequenced to confirm the identity of the rearrangement.

In similar methods, the invention can be used to identify an inversion. Such inversions may be pericentric inversions (involving the centromere) or peracentric inversions (involving an inversion of sequences on one arm of the chromosome). The identification of a test nucleic acid that hybridizes to both a first and to a second nucleic acid which normally are, for example, on opposite sides of a centromere, is an indication that the test nucleic acid includes the breakpoint of a chromosomal inversion. For example, a test nucleic acid that hybridizes to a portion of both chromosomal band 16p12 and 16q24 would be thought to contain an inversion that results in band 16p12 being brought into close proximity to band 16q24. As described above, the test nucleic acid should be sufficiently short such that it would not be expected to contain sequences corresponding to both the first and second nucleic acids, except as the result of a rearrangement. Likewise, a test nucleic acid that hybridizes to two, normally distant, portions of a chromosome or other nucleic acid also would be identified as containing a rearrangement. If desired, a test nucleic acid identified with these methods can be sequenced or mapped to confirm the presence and identity of the rearrangement.

Amplification Methods

Other particular embodiments of the invention make use of principles of boolean logic and of amplification techniques and nucleic acid labeling technology to label molecules with different moieties, such as biotin or digoxigenin (DIG) (Roche). For example, in particular methods of the invention, a portion of a first chromosome or other nucleic acid molecule is labeled with a first label, e.g., biotin, and a portion of a second chromosome or other nucleic acid molecule is labeled with a second label, e.g., DIG. The second label is distinct from the first label. The nucleic acids can be detectably labeled using conventional methods, as described infra. The labeled nucleic acids (e.g., chromosomal portions) are used as primers in nucleic acid synthesis, e.g., PCR, with a sample of nucleic acid containing the test nucleic acid (e.g., a patient's DNA) being used as a template. Preferably, the primers are randomly generated and thus a knowledge of the nucleotide sequence of the template is not required. For example, such primers can be generated by DOP-PCR or other random amplification methods which use as a template a nucleic acid comprising the sequence of all or a portion of a chromosome or other nucleic acid molecule.

A synthesized nucleic acid that includes both the first and second labeled primers (e.g., the biotin and DIG labels) is determined to be a hybrid molecule containing a portion of the first and second chromosomes, indicating that the test nucleic acid includes a breakpoint formed by a rearrangement between the first and second chromosomes or nucleic acid molecules. Portions of any number of chromosomes or nucleic acid molecules can be used as primers in such methods described herein, provided that distinct labels are used to identify hybrid molecules comprising portions of the particular chromosomes or nucleic acid molecules. If desired, a test nucleic acid identified as containing a rearrangement can subsequently be sequenced to confirm the presence and identity of the rearrangement.

In related methods, the first and second primers are derived from the same chromosome or nucleic acid molecule, and the amplification methods are used to identify rearrangements involving a single molecule (e.g., chromosome, such as an inversion, deletion, or fusion). The first and second primers may be specific to particular bands of a chromosome, e.g., by using band-specific nucleic acids as described herein, as primers for the amplification methods. For example, the first primer(s) may be specific to band 2p25, and the second primer(s) may be specific to band 2p11.2. Although the primer may be specific to a particular chromosomal band, it may otherwise be randomly generated (e.g., be by DOP-PCR using band-specific nucleic acids as the template). As described for the hybridization methods disclosed herein, the test nucleic acid is sufficiently short such that sequences corresponding to both primers would not be expected to be contained within a test nucleic acid except as the result of a rearrangement. If desired, the presence and identity of the rearrangement can be confirmed by DNA sequencing.

Kits

The invention thus also provides, in part, kits for detecting structural differences between nucleic acid molecules. In specific embodiments, kits of the invention contain instructions for performing methods for detecting structural differences between nucleic acid molecules. These methods will often be based on the detection of whether particular nucleic acid complexes are formed or fail to form. Various types of nucleic acid complexes which can be formed and detected using methods of the invention are described elsewhere herein.

Kits of the invention may contain any number of various components for practicing methods of the invention. One example of such a component is instructions for performing methods of the invention. Example of such instructions include those which direct individuals using the kits to perform methods for detecting structural differences between nucleic acid molecules.

As one skilled in the art would recognize, the full text of these instructions need not be included with the kit. One example of a situation in which kits of the invention would not contain such full length instructions is where directions are provided which inform individuals using the kits where to obtain instructions for using the kit. Thus, instructions for performing methods of the invention may be obtained from internet web pages, separately sold or distributed manuals or other product literature, etc. The invention thus includes kits which direct kit users to locations where they can find instructions which are not directly packaged and/or distributed with the kits. These instructions may be in any form including, but not limited to, electronic or printed forms.

### EXAMPLES

The invention is further illustrated by the following, non-limiting, examples.

In order to demonstrate the feasibility of the invention to detect a chromosomal translocation, the following examples made use of nucleic acids obtained from follicular lymphoma patients known to have a translocation between chromosome 14 and chromosome 18, viz. t(14;18) translocation (bcl2/Jh fusion). First, material from each chromosome was individually obtained either by employing a chromosome library, or from a preparation of flow sorted chromosomes. The latter method is advantageous as there would be no contaminating plasmid material and the entire chromosome would be represented. Flow sorted chromosomes 14 and 18 were obtained. The chromosome DNA was either extracted from the relevant chromosome library or used directly in the form of flow sorted chromosomes. This chromosomal material was then labeled with either biotin-16-dUTP (biotin dUTP) or DIG-11-dUTP (DIG dUTP) using either DOP-PCR (Roche) or any other random PCR thereby incorporating the labeled dUTP.

Since the human genome contains 3-6% repetitive sequences, it was desirable to rid the chromosome preparations of these repetitive and common sequences. The former was achieved using Cot 1 DNA (Roche), as it represents all alu and kpn family sequences. The Cot 1 DNA was immobilized on magnetic beads (Roche) and then used to extract the repetitive sequences in a solution of the chromosome preparations. What remained in the supernatant fluid (SNF) was representative of non-repetitive (not cot), labeled, chromosomal DNA.

The direct use of the labeled chromosome specific fragments as primers in a PCR reaction allowed the extension of these fragments across the chromosomal breakpoint, when a translocation was present. When two different chromosomes were involved in the translocation, the chimeric fragment of amplified DNA was labeled with both labels. This fragment was then isolated and/or detected by means of these two labels. By identifying the two involved chromosomes, this technique was used to characterize the chromosomal breakpoint. Thus, the invention can be used to isolate new or variant translocations and to identify the genes involved in disorders. A more detailed description of this example follows.

This example demonstrates that the methods of the invention could be used to identify specific DNA lesions without prior knowledge of the rearrangement. Chromosomes 14 and 18 were used is this example. Labeled chromosome 14 and 18 specific material was obtained by amplification of flow sorted chromosomes using degenerate oligonucleotide primed (DOP) PCR, and simultaneous labelling with biotin and Digoxigenin (DIG) respectively. Individual chromosomal material was purified by selectively eliminating all sequences homologous to Cot 1 DNA and sequences common to both chromosomes. The resultant specific chromosomal DNA was used as PCR primers to amplify DNA from negative controls and patients with Follicular Lymphoma bearing the t(14;18) translocation. DNA amplification occurring across the translocation breakpoint incorporated primers from both chromosomes 14 and 18 and thus also their respective tags viz. biotin and DIG. Molecules containing both the biotin and DIG moieties were selected using a solid support of streptavidin and then detecting with an anti-DIG alkaline phosphatase colour reaction, or vice versa. A positive reaction, identifying exclusively hybrid molecules (t(14;18)), was found in 3/3 patients and 0/3 controls. This example demonstrated that the methods of the invention could be used to identify specific chromosomal translocations.

The same methodology could be applied in a 2-dimensional matrix of paired chromosome-specific material covering all possible translocation partners and thus allowing analysis of any patient sample without the need for a preconception as to which pairs of chromosomes resulted in a translocation. The DNA spanning such breakpoints can be purified, e.g., using the unique combination of tags, to permit molecular characterization of unknown breakpoints. The materials and methods used in these examples are set forth below.

Flow sorted chromosomes were used in these examples. There were approximately 500 chromosomes per 33 μl, and 33 μl per 500 μl tube.

Chromosome 14 and 18 libraries were cultured overnight in 4-8 ml LB (Luria Broth) containing 80 μg/ml ampicillin, at 37° C. with agitation (Collins et al., Genomics 11:997-1006 (1991)). The media were either inoculated from the agar plates or 15% glycerol stocks using sterile techniques. The cells were pelleted in a microfuge (4° C.) for 5 minutes. A High Pure Plasmid Isolation Kit (Roche) was used to isolate the plasmid DNA. The DNA was then stored at −20° C. A 5.6 kb immunoglobulin probe (Jh) was also isolated in the above manner (Ravetch et al., Cell 27: 583-591(1981); Cleary et al., Science 81:593-597 (1984)).

Degenerate Oligonucleotide Primer (DOP) PCR can be used to allow the statistical and representative amplification of an uncharacterized or unknown DNA template. Typically, the reaction utilizes a universal primer possessing a 6-nucleotide long degenerate region (N⁶) that statistically represents all possible combinations of 6 nucleotides (kits for DOP PCR are commercially available, e.g., from Roche). The 3′ end of the primer may include a GC rich 6-nucleotide stretch, which theoretically occurs every 4 kb in the genome. This facilitates efficient primer hybridization and the start of the polymerization reaction. Typically, at the 5′ end of the DOP primer is a linker region containing a restriction site (e.g., XhoI), for possible cloning purposes (Table 1). Conventional techniques can be used in carrying out DOP PCR. Low specificity primer annealing was achieved in the first 5 cycles of DOP PCR through amplification at a relatively low annealing temperature of 30° C. Subsequent to primer annealing, the temperature was increased over a time range of 3 minutes to the polymerization temperature of 72° C. The amplification products of these low specificity cycles have the DOP primer sequence incorporated at their respective termini that are then utilized in the subsequent, more specific thermo-cycles (Telenius et al., Genomics 13:718-725 (1992)).

TABLE 1
Sequence of the DOP primers

| Primer | Sequence |
|---|---|
| DOP | 5′ ccg act cga gnn nnn nat gtg g 3′ |
| DOP2 | 5′ tgg cgg ccg cnn nnn nac gtc g 3′ |

A second, different DOP primer (DOP2 with a Not I restriction site) was designed and synthesized (Table 1). This was used on chromosome 18, while the commercial DOP primer was used on chromosome 14.

A second round of DOP PCR was used to incorporate biotin or DIG labels into the amplified chromosomes 14 and 18 respectively. The resulting combinations were as follows: Chromosome 14, amplified with DOP and labeled with biotin, and Chromosome 18, amplified with DOP2 and labeled with DIG.

DOP PCR was carried out using 33 μl of flow sorted chromosomes, which corresponded to approximately 500 chromosomes. Using a set of pipettes reserved for PCR only, to the tube containing the sorted chromosomes 14 or 18, the following was added: 3.75 μl of DOP or DOP2 primers (respectively), 37.5 μl DOP PCR kit master mix (containing nucleotides) (Roche, DOP PCR kit) and H₂O to a final volume of 75 μl. The mixture was processed in a Perkin Elmer 2400 thermal cycler using the following protocol: 94° C. for 9 minutes, 9 cycles of 94° C. for 1 minute, 30° C. for 1.5 minutes, ramping at 0.23° C./second to 72° C., 72° C. for 3 minutes, 30 cycles of 94° C. for 1 minute, 62° C. for 1 minute, 72° C. for 1 minute, and 72° C. for 9 minutes.

At this stage, there was a choice to generate fragments either 300-500 bp in length or 300-3000 bp in length by varying the cycling protocol (e.g., increasing the cycle times to increase the fragment length). The protocol generating shorter fragments was chosen, although the protocol generating longer fragments could be used. The primary PCR products were analyzed on a 2% agarose electrophoretic gel.

Labeled chromosome fragments, to be used as primers, were produced as follows. Using a set of pipettes reserved for PCR use only, the following components were added to a sterile 200 μl PCR tube: 14.5 μl of primary PCR product, 2.5 μl DOP (2 μM) or DOP2 (2 μM) primer, 200 μM of dCTP, dATP and dGTP, 66 μM of dTTP and 134 μM of labeled (biotin or DIG) dUTP (Roche). The mixture was vortexed, spun in a microfuge briefly and processed in a thermal cycler using the following protocol: 94° C. for 4 minutes, 35 cycles of 94° C. for 1 minute, 62° C. for 1 minute, 72° C. or 1 minute, and 72° C. for 9 minutes. The DOP PCR product was analyzed by electrophoresing 5-10 μl on a 2% agarose gel.

The labeled chromosome fragments were isolated as follows. Both chromosome products were passed through a Sephadex column (G50, molecular weight cut off >72 bp (Roche)) to remove all unincorporated, labeled nucleotides. The molecular weight cut off generally should be >50 bp, as these fragments serve as primers and probes at a later stage.

Removal of Repetitive Sequences

Optionally, repetitive sequences can be removed at this stage. The human genome contains 3-6% repetitive sequences; thus, these repetitive sequences preferably are removed from the chromosome preparations. Additionally, other regions of sequence homology between the two chromosomes can be removed, if desired.

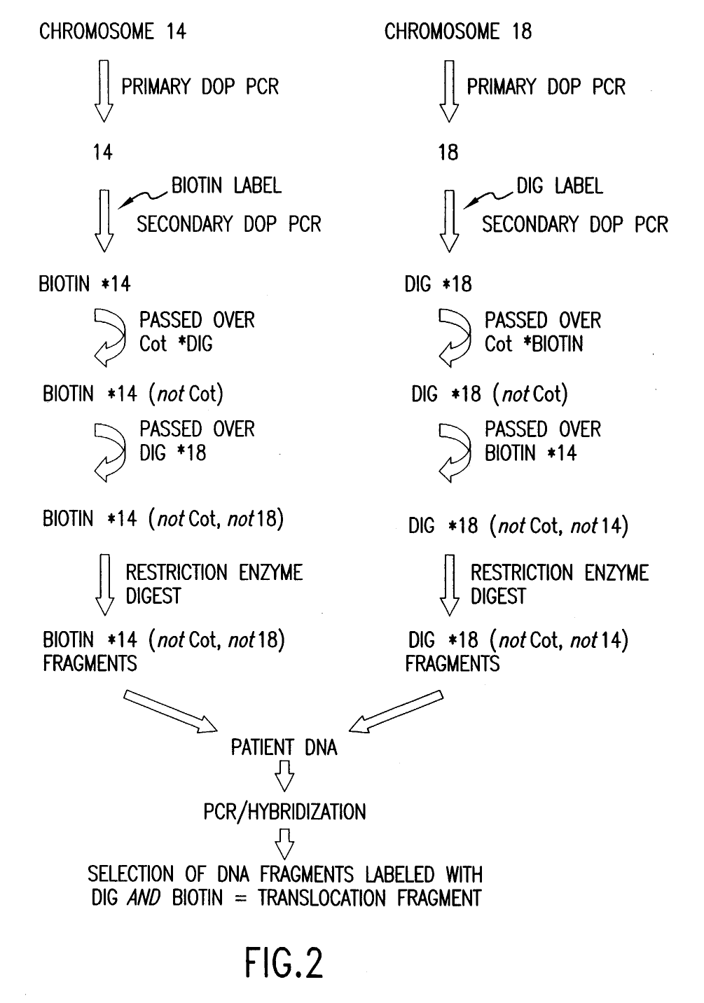

Six μg of Cot 1 (Cot) DNA (Roche), was randomly primed and labeled, using a random prime labelling kit (Roche), using conventional methods and either using biotin dUTP or DIG dUTP, in a 1:2 ratio with dTTP (FIG. 2). The labeled Cot DNA was then passed over a Sephadex G50 column to remove the unincorporated nucleotides. Cot 1 DNA in excess rapidly hybridizes to repetitive sequences in the target molecules, allowing most of the specific sequences to remain single stranded and to then bind to their chromosomal targets.

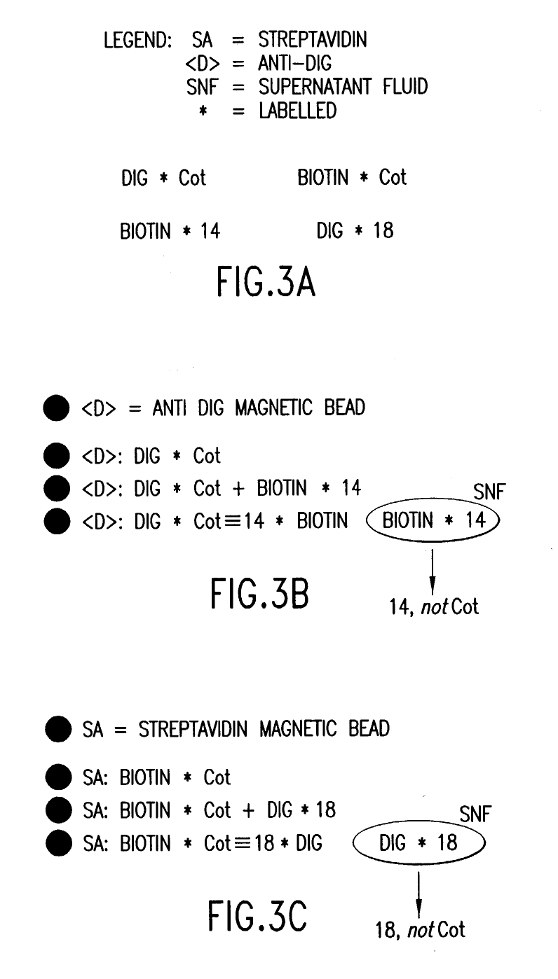

The biotin labeled Cot DNA was immobilized onto streptavidin magnetic beads (Roche) and used to ‘fish-out’ the repetitive sequences in the DIG labeled chromosome 18 preparation (FIGS. 2 and 3A-3C). What remained in the supernatant was representative of non-repetitive (not cot), labeled, chromosomal DNA. The kit manufacturer's protocol (Roche) was followed with a few modifications. Two hundred μl of streptavidin coated magnetic beads were washed twice with TEN₁₀₀ buffer (10 mM Tris-HCl, 1 mM EDTA, 0.1M NaCl, pH 7.5) and placed in a final volume of 200 μl TEN₁₀₀. The labeled Cot DNA was denatured at 94° C. for 5 minutes, flash cooled on ice and made up to a volume of 100 μl with TEN₁₀₀. The Cot DNA was added to the magnetic beads and left at room temperature for 30 minutes in order to allow the streptavidin and biotin to associate. The magnetic beads were separated on a magnetic stand/separator (Roche), the supernatant fluid removed and the beads washed well with TEN₅₀₀ (as above but with 0.5M NaCl) and placed in a 200 μl volume of TEN₅₀₀ (off the magnetic separator). Other conventional techniques for isolating labeled DNA can be used in the alternative.

A 200 μl aliquot of the DIG labeled chromosome 18 was denatured at 94° C., flash cooled on ice and added to the streptavidin magnetic beads, biotin labeled Cot mixture and placed at room temperature for 30 minutes to allow all Cot-like sequences in the chromosome 18 preparation to associate with the biotin labeled Cot. The magnetic beads were again separated on a magnetic stand and the supernatant fluid removed into a clean tube. This supernatant fluid was now largely free of repetitive sequences and could be termed chromosome 18 not Cot.

The procedure was repeated for biotin labeled chromosome 14, using DIG labeled Cot and anti-DIG (<DIG>) magnetic beads, otherwise the protocol remained exactly the same. At all stages, the fraction bound to the magnetic beads could be removed with heat (94° C.) and pure H₂O to dissociate the hybridized chromosomes 14 and 18; and 6M guanidine HCl could be used to break the bonds between the streptavidin and biotin or the anti-DIG and DIG moieties. These fractions were spotted onto a nylon membrane and the label was detected, enabling one to confirm whether that the correct fraction had bound.

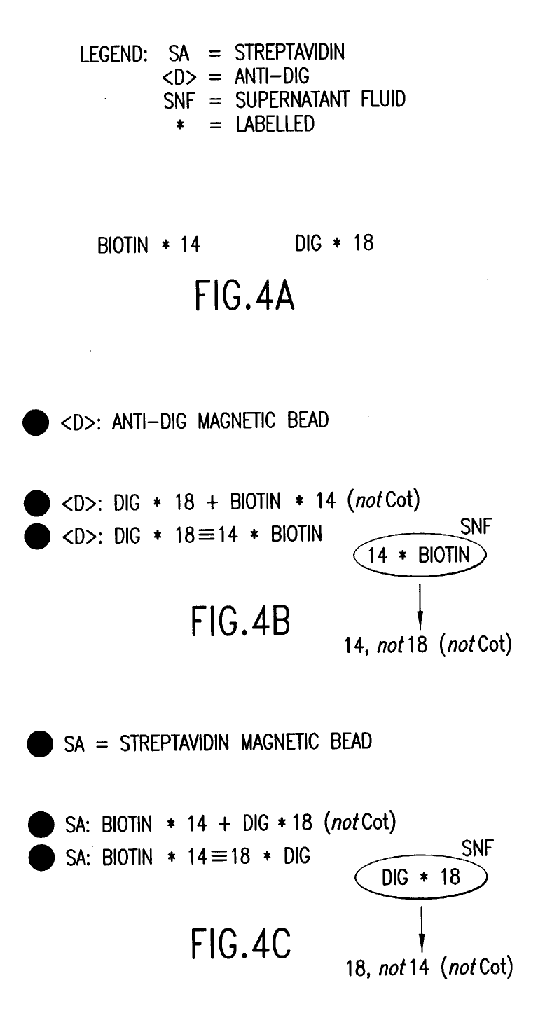

Optionally, sequences common to both chromosomes can be removed (FIGS. 4A-4C). In this example, an aliquot (one third of the total volume) of DIG labeled chromosome 18 (not Cot) was immobilized on <DIG> magnetic beads and used to ‘fish out’ common sequences from the biotin labeled chromosome 14 (not Cot). The same was done for the remaining two thirds of DIG labeled chromosome 18, by immobilizing an aliquot of biotin labeled chromosome 14 (one third) on streptavidin magnetic beads. The whole procedure can be repeated if desired. The resulting SNFs were termed: DIG labeled chromosome 18, not Cot, not 14 and Biotin labeled chromosome 14, not Cot, not 18 (FIGS. 2 and 4A-4C) The SNFs were then precipitated with 10% (v/v) 3M NaOH, pH 5.2 and 2.5 (v/v) 100% ethanol, at −70° C.

Preferably, the chromosomal fragments produced by DOP PCR are digested with restriction enzymes to produce 3′ ends that are complementary to chromosomal sequences. In this example, the generation of preferred 3′ ends was achieved by digesting the PCR products with frequent (tetranucleotide) cutters (Rsa I and/or Hind III) or hexanucleotide cutters, thus generating smaller fragments having 3′ hydroxyl ends complementary to chromosomal DNA and thus the patient's DNA. Assuming that restriction endonuclease sites are distributed randomly along the DNA, a tetranucleotide target can be expected to occur once every 4⁴ (256) nucleotides and a hexanucleotide target every 4⁶ (4096) nucleotides.

The labeled chromosome material was resuspended in 8 μl of H₂O, 1 μ(10U) of enzyme and 1 μl enzyme-buffer was added, and the mixture was placed at 37° C. overnight.

The test nucleic acids (DNA or RNA) used in the methods of the invention can be derived from any source suspected of containing a chromosomal translocation. Conventional methods can be used to isolate such nucleic acids. In this example, follicular lymphoma patients were chosen who were known to have a classical t(14:18) translocation, as defined by classical t(14:18) PCR and oligo extension, and additionally one patient (3) had been shown cytogenetically to have a t(14;18) translocation. The normal samples were negative using t(14;18) PCR. Patients were also shown to have a monoclonal band with the IgH PCR.

DNA was extracted from paraffin embedded tissue, blood or marrow of the patients with follicular lymphoma (t(14:18)) and known normal controls according to standard methods. The DNA concentration was analyzed spectrophotometrically, and the DNA was run on a 2% agarose gel.

Identification of a Chromosomal Translocation

The invention provides methods for identifying a chromosomal translocation. One embodiment involves PCR technology, whereas an alternative embodiment involves hybridization technology.

In this example, identification of the chromosomal translocation by using PCR technology was carried out as follows. The t(14;18) translocation was isolated by PCR amplification of the patient DNA with the two sets of labeled primers generated as described above. In principle, the sets of primers should randomly amplify all areas of chromosomes 14 and 18. However, only where there is a translocation between the two chromosomes will the resulting molecule be a product of both primers and thus have incorporated both biotin and DIG labels.

A PCR reaction was made up with 1.0 μg of patient (or normal) DNA, 0.1 μg, of DIG labeled chromosome 18 and 0.1 μg of biotin labeled chromosome 14 as primers, 0.2 μM of dNTPs, 1.25U of Taq DNA polymerase (Roche) or 0.25 μl Expand high fidelity mixture (Roche), and 1.5 mM MgCl₂. The reaction was carried out at: 94° C. for 4 minutes; 40 cycles at: 94° C. for 1 minute, 62° C. for 1 minute, 72° C. for 1 minute, and 94° C. for 7 minutes. The PCR products were run on a 2% agarose gel confirm that amplification had occurred, as indicated by a smear.

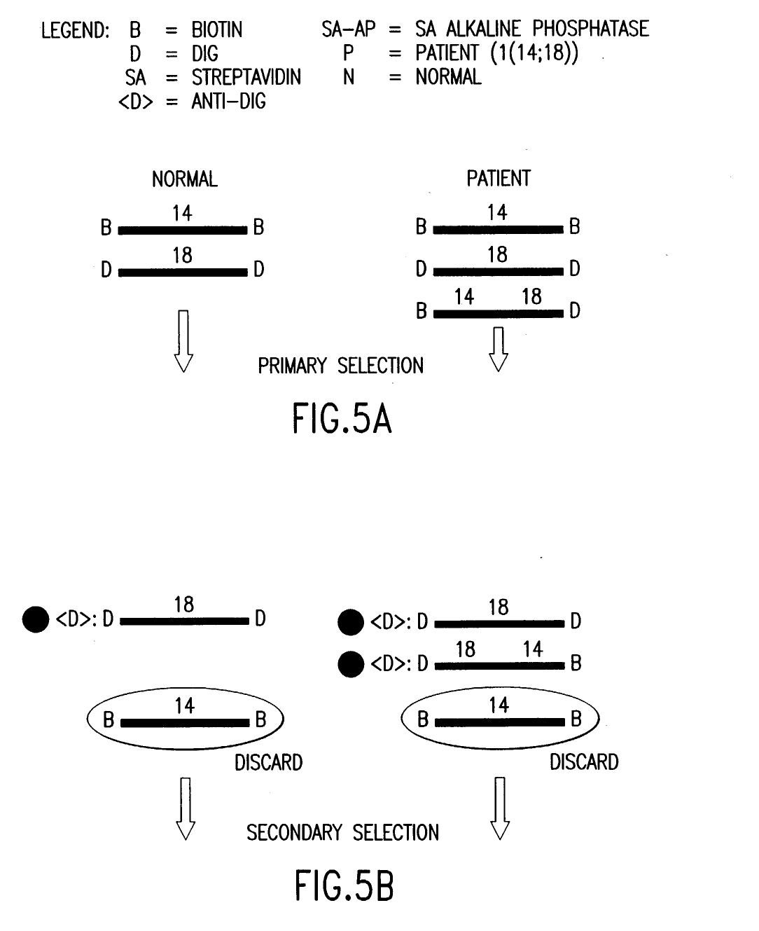

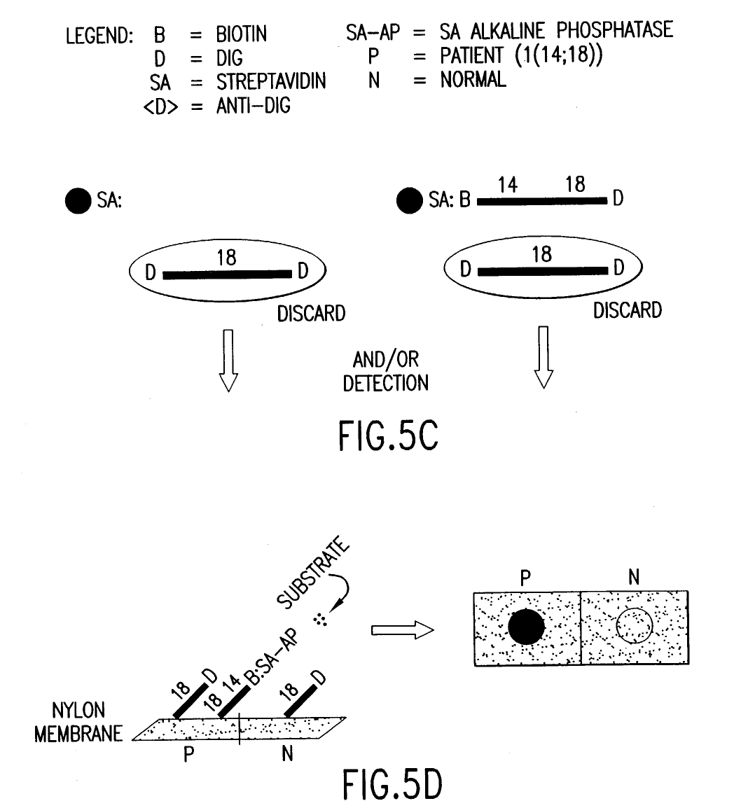

This PCR product was purified using magnetic beads either coated with <DIG> or streptavidin. The <DIG> beads used first selected out all PCR products that had incorporated DIG, and biotin-only labeled products were discarded (primary selection) (FIGS. 5A-5D). The <DIG> selected products were then eluted from these beads (the fraction bound to the <DIG> magnetic beads can be removed with heat (94° C.) and pure H₂O), and the products added to the streptavidin beads which would select out all PCR products that had biotin incorporated (secondary selection). Thus, only the hybrids nucleic acids containing both DIG and biotin were isolated (since the biotin-only products had already been discarded). In an alternative method, the primary selection was carried out as described, and the secondary selection occurred in situ as a color reaction using streptavidin alkaline phosphatase. This alternative method used NBT/BCIP (Roche), which formed a blue/purple color precipitate only when the translocation was present (FIGS. 5A-5D).

Dot Blotting

All samples that needed to be visualized, including those required to check the binding and elimination processes, were reduced in volume (ethanol precipitation), and approximately 5 μl was spotted onto a nylon membrane. The sample was allowed to air dry and was UV-nicked (320 nm, 45 seconds) to fix the DNA to the membrane. The presence of either DIG or biotin was then detected using either <DIG> or streptavidin alkaline phosphatase and NBT/BCIP (Roche) using conventional techniques. A blue/purple color precipitate was formed only when the correct moiety was present.

Microtiter Plate (MTP) Analysis

Alternatively to being blotted on a membrane, the hybrid PCR products were isolated in wells of a microtiter plate having streptavidin or <DIG> MTP attached to the surface of the well. Thus, microtiter plates replaced the magnetic beads, described above, in the primary and secondary selection processes. The detection step took place in the MTP using <DIG> or streptavidin horseradish peroxidase (HRP) and ABTS (Roche). A soluble substrate was detected at 405 nm (reference filter, 492 nm), at 15 minutes, in the MTP reader. When a <DIG> MTP was used to capture the hybrid PCR products, the presence of the hybrid molecule was detected with streptavidin HRP. When a streptavidin MTP was used, detection was carried out using <DIG> HRP (Roche kit).

Hybridization

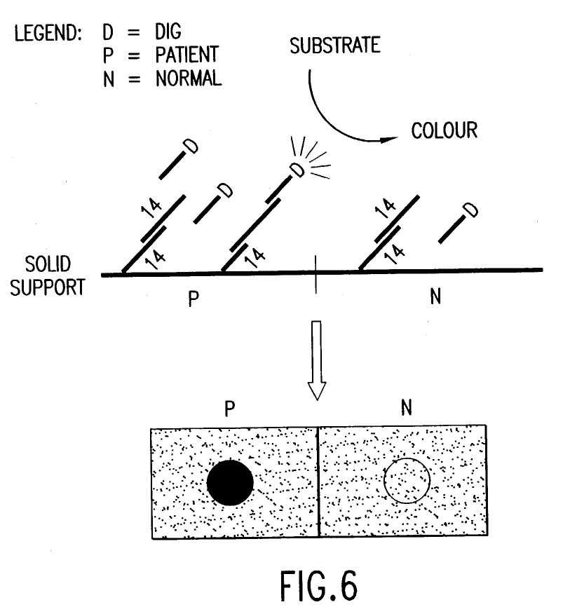

In an alternative method, hybridization techniques were used to identify the chromosomal translocation (FIG. 6). This method does not require PCR amplification as described above. This method relies on the hybridization of a test nucleic acid containing the translocation to a fragment of a first chromosome (e.g., chromosome 14) which is immobilized onto a solid support, e.g., by virtue of a biotin label. The test nucleic acids also were hybridized to labeled fragments of a second chromosome (e.g., chromosome 18 fragments (‘primers’ generated as described above)). The presence of the resulting trimolecular sandwich was then detected by colorimetric means. Only test nucleic acids containing nucleic acids from both the first and second chromosomes (both chromosomes 14 and 18) resulted in a positive signal.

In lieu of using an entire chromosome (e.g., all of chromosome 18), a section of the chromosome thought to encompass all relevant translocations described in that area can be used, e.g., a DIG-labeled immunoglobulin gene probe (Jh). The use of a portion of the chromosome results provides a more simplified assay.

Results

Test nucleic acids obtained from patients (n=5) and normal controls (n=4) were checked for their monoclonal (or lack thereof) status using the t(14;18) PCR and oligo-extension, and/or the IgH gene rearrangement PCR. All five patients showed a positive bcl2/Jh result with PCR and subsequent oligo-extension, confirming that the patients were all positive for the t(14;18) translocation. Patients 1, 2, 3 and 4 showed monoclonal B-cell populations. Patient 5 was not analyzed (as the patient is positive for the t(14;18) PCR, this is not a concern). The controls samples were negative for the above t(14;18) PCR.

Optionally, the labeled chromosomal fragments were analyzed as follows. On gels containing flow sorted chromosome 14 and 18 fragments following DOP PCR, a smear can be seen between 200 and 2000 bp. At each selection step, aliquots of captured and supernatant fluid fractions were spotted onto nylon membranes in duplicate and were analyzed to determine which label was present (DIG, biotin or both) throughout the repetitive sequence selection process described above. There was no cross contamination visible, and the results corresponded to the expected labels. Other conventional methods for confirming the identity of purity of the PCR amplified nucleic acids can be substituted, if desired.

In this example, patient 1 was positive after both primary and secondary selections and normal 1 remained negative. All three patients (1, 2 and 3) were positive, and normal 2 appeared to be negative. Dot blot analysis indicated that patient 4 had a positive signal compared to normal 3. Thus a positive reaction, identifying exclusively hybrid molecules (t(14;18)), was obtained in 4 of the 4 patients and in 0 of the 3 normal controls. Using the hybridization method with the Jh probe, a positive signal was obtained with patient 5, and no signal was obtained with normal 2.

Microtiter Plate (MTP) Readings

The results obtained using soluble color reactions and a MTP reader also demonstrate that the method of the invention can be used to identify nucleic acids containing a chromosomal translocation. Signals approximately twice that of normal 4 were obtained with test nucleic acids derived from a patient (Table 2). The positive and negative controls were all within the expected range. These results confirm that the methods of the invention were able to repeatedly detect translocations in a variety of forms.

Table 2: MTP readings for the mictotiter well representing patient 1 compared to a normal control (N4). These readings were obtained using <DIG> MTP for the primary selection (1°) and detecting the hybrid molecule with streptavidin HRP at 405 nm at 15 minutes after substrate addition. The secondary selection (2°) was performed on a streptavidin MTP, and the readings were obtained using <DIG> HRP.

| Selection | Kit Positive | Kit negative | Patient (well) | Normal (well) |
|---|---|---|---|---|
| 1° | 0.353 | 0.014 | 0.468 | 0.112 |
| 2° | 0.375 | 0.008 | 0.233 | 0.064 |

Summary

The examples set forth above demonstrate that the methods of the invention provide for the identification of chromosomal translocations in a test nucleic acid (e.g., DNA obtained from a patient). Thus, the disclosed methods can be used to identify known and novel chromosomal translocations.

Two-Dimensional Matrix

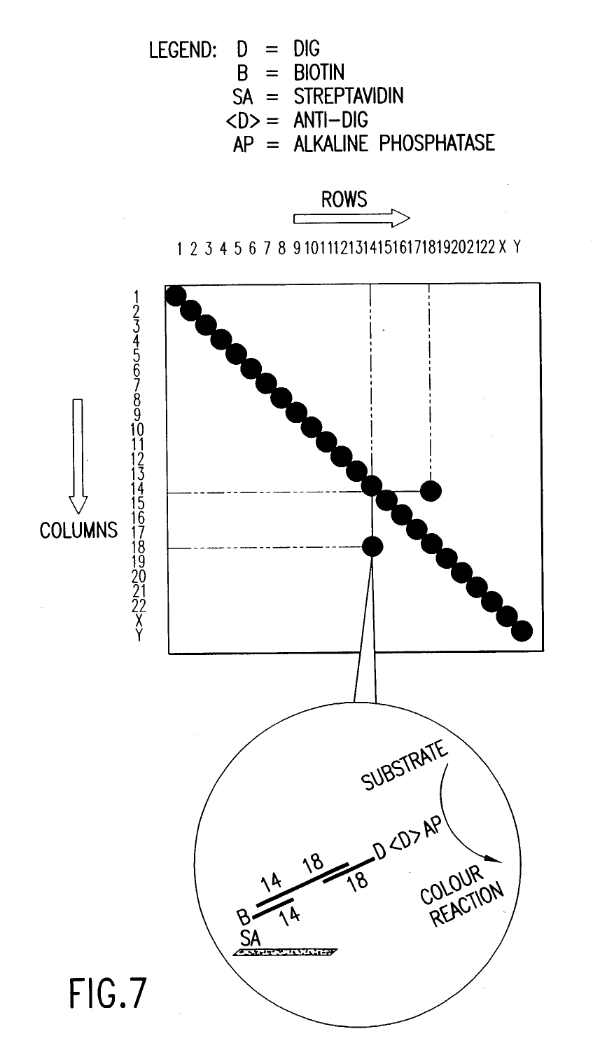

The methods of the present invention can readily be used in the context of a two-dimensional array. For example, two panels of differentially labeled chromosomes can be used: panel 1: chromosomes 1-22, X and Y labeled with biotin; and panel 2: chromosomes 1-22, X and Y labeled with DIG placed in an array. The presence of a chromosomal translocation can be detected by first immobilizing a biotin-labeled panel of chromosomal DNA (chromosomes 1-22, X, and Y) onto a streptavidin-tagged solid support, one ‘column’ for each biotin labeled-chromosome (FIG. 7 (↓)). These solid supports are then exposed to the test nucleic acid, e.g., patient DNA. Each immobilized chromosome binds to the relevant patient DNA as well as to any hybrid DNA containing material homologous to that chromosome. The immobilized chromosomes and the captured DNA would then each be further hybridized to a DIG-labeled panel of chromosomal DNA (chromosome 1-22, X and Y), one ‘row’ for each DIG-labeled chromosome (FIG. 7 (→)), generating a matrix or array of 576 potential points. At points where both the biotin-labeled chromosome (↓) and the DIG-labeled chromosome (→) are the same, a signal is generated, and thus serves as a convenient experimental control. A signal obtained representing two different labels and thus two different chromosomes indicates the presence of a translocation involving those two chromosomes (FIG. 7, insert). Such a two-dimensional array provides for the detection of essentially any translocation between two chromosomes. After identifying the two chromosomes involved in a translocation, the sequence of the chromosomal breakpoint can be characterized using conventional techniques to identify the genes involved (e.g., genes causing the neoplasm). For example, the isolated DNA can be released from the solid support and cloned into a vector, e.g., for subsequent sequencing. In similar methods, a two0dimensional array can be used to identify chromosomal rearrangements within a chromosome. In such methods, portions of the chromosome (e.g., band-specific nucleic acids) are used as the first and second nucleic acids in the hybridization methods, as described infra, thus forming a matrix that utilizes portions of a chromosome.

What is claimed is:

1. A method for identifying a chromosomal rearrangement in a test nucleic acid, the method comprising:
providing a substrate having attached thereto a first nucleic acid comprising the sequence of all or a portion of a first chromosome;
contacting said substrate with a test nucleic acid under conditions that permit a first portion of the test nucleic acid to hybridize to all or a portion of said first nucleic acid;
contacting the test nucleic with a second nucleic acid under conditions that permit hybridization of all or a portion of the second nucleic acid to a second portion of the test nucleic acid, wherein the second nucleic acid comprises the sequence of all or a portion of a second chromosome; and
detecting hybridization of said test nucleic acid to each of said first nucleic acid and said second nucleic acid as an indication that the test nucleic acid comprises a chromosomal rearrangement.

2. The method of claim 1, wherein said chromosomal rearrangement comprises a chromosomal translocation.

3. The method of claim 1, wherein said first nucleic acid is detectably labeled with a label.

4. The method of claim 1, wherein said second nucleic acid is detectably labeled with a label.

5. The method of claim 1, wherein said first nucleic acid is detectably labeled with a first label;
further wherein said second nucleic acid is detectably labeled with a second label and wherein said second label is distinct from said first label.

6. The method of claim 5, wherein detecting hybridization of said test nucleic acid to each of said first nucleic acid and said second nucleic acid comprises detecting the colocalization of said first and said second labels.

7. The method of claim 1, wherein said substrate is selected from the group consisting of a microchip, a microtiter plate, plastic, a nylon membrane, glass, chromatography resin, and a bead.

8. The method of claim 1, wherein said test nucleic acid comprises DNA.

9. The method of claim 1, wherein said test nucleic acid comprises RNA.

10. The method of claim 1, wherein said test nucleic acid is derived from a human cell.

11. The method of claim 10, wherein said human cell is a primary human cell.

12. The method of claim 10, wherein said human cell is a cell of a human cell line.

13. The method of claim 3, wherein said label is selected from the group consisting of an enzymatic label, a fluorescent label, a radioisotopic label, and a chemiluminescent label.

14. The method of claim 4, wherein said label is selected from the group consisting of an enzymatic label, a fluorescent label, a radioisotopic label, and a chemiluminescent label.

15. The method of claim 1, wherein said portion of said first and/or said second chromosome comprises about 100 nucleotides to about 300,000 nucleotides.

16. The method of claim 10, wherein said portion of said first and/or said second chromosome comprises about 200 to about 3000 nucleotides.

17. The method of claim 1, wherein said test nucleic acid comprises about 200 to about 5000 nucleotides.

18. The method of claim 1, wherein said substrate has attached thereto a population of distinct first nucleic acids, wherein each first nucleic acid comprises the sequence of all or a portion of a chromosome, and wherein the population of first nucleic acids comprises the sequences of all or a portion of a plurality of chromosomes.

19. The method of claim 1, wherein said test nucleic acid is contacted with a population of distinct second nucleic acids, wherein each second nucleic acid comprises the sequence of all or a portion of a chromosome, and wherein the population of second nucleic acids comprises the sequences of all or a portion of a plurality of chromosomes.

20. The method of claim 18, wherein said substrate comprises a plurality of subsections, each subsection having attached thereto a distinct first nucleic acid of said population.

21. The method of claim 18, wherein said substrate has attached thereto a population of at least 10 distinct first nucleic acids.

22. The method of claim 1, wherein said chromosomal rearrangement is indicative of a cancer.

23. The method of claim 22, wherein said test nucleic acid comprises a breakpoint of a chromosomal translocation selected from the group consisting of t(14;18), t(8;21), t(9;22), t(15;17), t(8;14), t(11;14), t(9;14), t(5;14), t(1;14), t(3;14), t(14;15), t(1;14), t(4;14), t(6;14), t(14;16 t(2;5), t(14;15), t(3;8), t(9;16), t(3;5), t(2;13), and t(11;22).

24. The method of claim 22, wherein said cancer is a leukemia.

25. The method of claim 22, wherein said cancer is a lymphoma.

26. A method for diagnosing cancer or a predisposition to cancer in a patient, the method comprising identifying a chromosomal rearrangement according to the method of claim 1, wherein said test nucleic acid is derived from said patient and comprises a chromosomal rearrangement.

27. A method for determining the prognosis, progression, or treatment protocol of a cancer in a patient, the method comprising identifying a chromosomal rearrangement according to the method of claim 1, wherein said test nucleic acid is derived from said patient and comprises a chromosomal rearrangement.

28. The method of claim 26, wherein the chromosomal rearrangement is selected from the group consisting of t(14;18), t(8;21), t(9;22), t(15;17), t(8;14), t(11;14), t(9;14), t(5;14), t(1;14), t(3;14), t(14;15), t(1;14), t(4;14), t(6;14), t(14;16), t(2;5), t(14;15), t(3;8), t(9;16), t(3;5), t(2;13), and t(11;22).

29. A trimolecular sandwich comprising:
a substrate;
a first nucleic acid comprising the sequence of all or a portion of a first chromosome, wherein said first nucleic acid is attached to said substrate;
a test nucleic acid, wherein a first portion of said test nucleic acid is hybridized to all or a portion of said first nucleic acid; and
a second nucleic acid, wherein all or a portion of the second nucleic acid is hybridized to a second portion of the test nucleic acid, and wherein the second nucleic acid comprises the sequence of all or a portion of a second chromosome.

30. The trimolecular sandwich of claim 29, wherein said substrate has attached thereto a population of distinct first nucleic acids, wherein each first nucleic acid comprises the sequence of all or a portion of a chromosome, and wherein the population of first nucleic acids comprises the sequences of all or a portion of a plurality of chromosomes.

31. The trimolecular sandwich of claim 29, wherein the trimolecular sandwich comprises a population of distinct test nucleic acids.

32. The trimolecular sandwich of claim 29, wherein said trimolecular sandwich comprises a population of second nucleic acids, wherein each second nucleic acid comprises the sequence of all or a portion of a chromosome, and wherein the population of second nucleic acids comprises the sequences of all or a portion of a plurality of chromosomes.

33. The trimolecular sandwich of claim 29, wherein said test nucleic acid comprises the breakpoint of a chromosomal translocation selected from the group consisting of t(14;18), t(8;21), t(9;22), t(15;17), t(8;14), t(11;14), t(9;14), t(5;14), t(1;14), t(3;14), t(14;15), t(1;14), t(4;14), t(6;14), t(14;16), t(2;5), t(14;15), t(3;8), t(9;16), t(3;5), t(2;13), and t(11;22).

34. A method for identifying a test nucleic acid comprising a chromosomal rearrangement, the method comprising:
providing a test nucleic acid as a template for nucleic acid synthesis;
contacting said test nucleic acid with each of (i) a population of first primers, wherein substantially all of said first primers are detectably labeled with a first label and wherein said population of first primers is randomly generated, and wherein each of said first primers hybridizes to a portion of a first chromosome and (ii) a population of second primers, wherein substantially all of said second primers are detectably labeled with a second label and wherein said population of second primers is randomly generated, and wherein each of said second primers hybridizes to a portion of a second chromosome, and further wherein said second label is distinct from said first label;
synthesizing a nucleic acid complementary to said test nucleic acid, wherein said test nucleic acid serves as a template for nucleic acid synthesis and one of said first primers and one of said second primers prime synthesis of the synthesized nucleic acid; and
detecting a synthesized nucleic acid comprising each of said first and said second labels as an indication that the test nucleic acid comprises a chromosomal rearrangement.

35. The method of claim 34, further comprising isolating said synthesized nucleic acid comprising each of said first and said second labels.

36. The method of claim 35, wherein said synthesized nucleic acid is isolated by affinity chromatography.

37. A kit comprising:
(i) a substrate, the substrate having attached thereto a first nucleic acid comprising all or a portion of a first chromosome, wherein the first nucleic acid is detectably labeled with a first label; and
(ii) a second nucleic acid comprising all or a portion of a second chromosome, wherein the second nucleic acid is detectably labeled with a second label and wherein said second label is distinct from said first label.

38. The kit of claim 37, wherein said substrate has attached thereto a population of distinct first nucleic acids, wherein each said first nucleic acid comprises the sequence of all or a portion of a chromosome, and wherein the population of first nucleic acids comprises the sequences of all or a portion of a plurality of distinct chromosomes.

39. The kit of claim 37, wherein said kit comprises a population of second nucleic acids, wherein each said second nucleic acid comprises the sequence of all or a portion of a chromosome, and wherein the population of second nucleic acids comprises the sequences of all or a portion of a plurality of chromosomes.

40. A kit comprising:
(i) a substrate
(ii) a nucleic acid comprising all or a portion of a chromosome, and
(ii) instructions for using said substrate and said nucleic acid to detect a chromosomal rearrangement.

41. The kit of claim 40, wherein said nucleic acid is attached to said substrate.

42. The kit of claim 40, wherein said nucleic acid is detectably labeled.

43. The kit of claim 40, further comprising a second nucleic acid comprising all or a portion of a chromosome.

44. The kit of claim 43, wherein said second nucleic acid is detectably labeled.

45. The kit of claim 40, wherein said chromosomal rearrangement comprises a chromosomal translocation.

46. A method for identifying a chromosomal rearrangement in a test nucleic acid, the method comprising:
providing a substrate having attached thereto a first nucleic acid comprising the sequence of a first portion of a chromosome;
contacting said substrate with a test nucleic acid under conditions that permit a first portion of the test nucleic acid to hybridize to all or a portion of said first nucleic acid;
contacting the test nucleic with a second nucleic acid under conditions that permit hybridization of all or a portion of the second nucleic acid to a second portion of the test nucleic acid, wherein the second nucleic acid comprises the sequence of a second portion of said chromosome; and
detecting hybridization of said test nucleic acid to each of said first nucleic acid and said second nucleic acid as an indication that the test nucleic acid comprises a chromosomal rearrangement.

47. The method of claim 46, wherein said chromosomal rearrangement comprises a chromosomal inversion.

48. The method of claim 46, wherein said chromosomal rearrangement comprises a deletion.

49. The method of claim 46, wherein said first nucleic acid is detectably labeled with a label.

50. The method of claim 46, wherein said second nucleic acid is detectably labeled with a label.

51. The method of claim 46, wherein said first nucleic acid is detectably labeled with a first label;
further wherein said second nucleic acid is detectably labeled with a second label and wherein said second label is distinct from said first label.

52. The method of claim 46, wherein detecting hybridization of said test nucleic acid to each of said first nucleic acid and said second nucleic acid comprises detecting the colocalization of said first and said second labels.

53. The method of claim 46, wherein said first nucleic acid hybridizes to all or a portion of a first chromosomal band, and wherein said second nucleic acid hybridizes to all or a portion of a second chromosomal band.

54. The method of claim 46, wherein said substrate has attached thereto a population of distinct first nucleic acids, wherein each first nucleic acid comprises the sequence of a portion of a chromosome, and wherein the population of first nucleic acids comprises the sequences of a plurality of portions of said chromosome.

55. The method of claim 46, wherein said test nucleic acid is contacted with a population of distinct second nucleic acids, wherein each second nucleic acid comprises the sequence of a portion of a chromosome, and wherein the population of second nucleic acids comprises the sequences of a plurality of portions of said chromosome.

56. The method of claim 46, wherein said substrate comprises a plurality of subsections, each subsection having attached thereto a distinct first nucleic acid of said population.

57. The method of claim 46, wherein said substrate has attached thereto a population of at least 10 distinct first nucleic acids.

58. The method of claim 46, wherein said substrate has attached thereto a population of at least 20 distinct first nucleic acids.

59. A method for comparing a test nucleic acid with a reference nucleic acid, the method comprising:
providing a substrate having attached thereto a first nucleic acid comprising the sequence of a first portion of a first chromosome;
contacting said substrate with a test nucleic acid under conditions that permit a first portion of the test nucleic acid to hybridize to all or a portion of said first nucleic acid;
contacting the test nucleic with a second nucleic acid under conditions that permit hybridization of all or a portion of the second nucleic acid to a second portion of the test nucleic acid, wherein the second nucleic acid comprises the sequence of (i) a second portion of said chromosome or (ii) a portion of a second chromosome;
detecting hybridization of said test nucleic acid to each of said first nucleic acid and said second nucleic acid; and
comparing the ability of said test nucleic acid to hybridize to each of said first nucleic acid and said second nucleic acid with the ability of said reference nucleic acid to hybridize to each of said first nucleic acid and said second nucleic acid.

60. The method of claim 59, wherein said test nucleic acid and said reference nucleic acid are derived from different species.

61. The method of claim 59, wherein said test nucleic acid and said reference nucleic acid are derived from a single species.

62. The method of claim 59, wherein said test nucleic acid and said reference nucleic acid are derived from a single-cell organism.

63. The method of claim 59, wherein said test nucleic acid and said reference nucleic acid are derived from a multicellular organism.


SEQUENCE LISTING (2 sequences)

SEQ ID NO: 1 - DNA, 22 bp, Artificial Sequence
  DOP Primer
ccgactcgagnnnnnnatgtgg

SEQ ID NO: 2 - DNA, 22 bp, Artificial Sequence
  DOP Primer
tggcggccgcnnnnnnacgtcg